(19) United States
(12) Patent Application Publication
(10) Pub. No.: US 2010/0262434 A1
(43) Pub. Date: Oct. 14, 2010

(54) METHOD AND APPARATUS TO CALCULATE DIABETIC SENSITIVITY FACTORS AFFECTING BLOOD GLUCOSE

(76) Inventor: Steven A. Shaya, Austin, TX (US)

Correspondence Address: PAUL AND PAUL, 2000 MARKET STREET, SUITE 2900, PHILADELPHIA, PA, 19103, US

(21) Appl. No.: 12/742,120

(22) PCT Filed: Sep. 22, 2008
(86) PCT No.: PCT/US08/77176

§ 371 Date: May 10, 2010

Related U.S. Application Data
(60) Provisional application No. 61/013,410, filed on Dec. 13, 2007.

(51) Int. Cl. A61B 5/00

(52) U.S. Cl. 705/3

(57) ABSTRACT

Methods and apparatus are provided for determining a diabetic patient's carbohydrate to insulin ratio (CIR), carbohydrate to blood glucose ratio (CGR), and insulin sensitivity factor (ISF) using the patient's record of blood glucose readings, carbohydrate consumption and insulin doses. The method provides the sensitivity factors that best account for the patient's observed blood glucose changes by linear regression of appropriately transformed variables. An apparatus that can collect and store the blood glucose readings, insulin dosages, and carbohydrate intake data and process these data according to this invention can generate statistically characterized sensitivity factors to advise the diabetic patient on optimal bolus insulin dosages.

### BACKGROUND OF THE INVENTION

1. Field of the Invention

The invention relates generally to methods and apparatus to calculate the sensitivity factors used to set insulin dosage for a diabetic patient to treat high or low glucose or to prevent hyperglycemia when consuming food. The methods can be incorporated into blood glucose meters, insulin pumps, or computer programs to determine personal sensitivity factors and their statistical uncertainty.

2. Brief Description of the Prior Art

Diabetic patients of Type 1 and many times, Type 2 diabetes as well, must manage their blood glucose concentration with injections of insulin multiple times a day because their pancreas is not capable of producing adequate insulin which is necessary to support glucose metabolism. In Type 1 diabetes, the pancreas cannot supply normal levels of insulin and in Type 2 diabetes there is a combination of problems starting with a need for excess levels of insulin to overcome insulin resistance. In some cases, this can lead to a decline in pancreatic insulin output capacity. The goal of administrating the proper insulin dose is to maintain blood glucose concentrations close to the physiological norm, which is around 1 gram of glucose per liter of blood. This normal target is commonly expressed as the equivalent 100 mg/dL.

The intention of administrating multiple insulin doses per day is to normalize blood glucose after meals. Major studies, sponsored by the National Institute of Diabetes and Digestive and Kidney Diseases (NIDDK), have proven that keeping blood sugar levels as close to normal as possible reduces the risk of developing the major complications of diabetes. In 1993, and in subsequent follow-up results, clinical studies have shown tight control of the blood glucose levels of diabetic patients reduces serious complications that arise over time, such as heart disease, kidney disease, amputations, and blindness. [The effect of intensive treatment of diabetes on the development and progression of long-term complications in insulin-dependent diabetes mellitus, The Diabetes Control and Complications Trial Research Group, New England Journal of Medicine, 329:977-986 (1993); American Diabetes Association (ADA). Standards of medical care in diabetes. VI. Prevention and management of diabetes complications, Diabetes Care; 30 (January 2007); Supplement 1:S15-24.]

Using insulin to prevent hyperglycemia requires great precision. If not enough insulin is administered, the blood glucose level will be hyperglycemic, leading to adverse health complications. If too much insulin is administered, glucose levels will fall significantly below normal, creating a serious acute condition called hypoglycemia. It is a problem for a diabetic patient to know his of her immediate requirement for insulin. Even using insulin, it is not uncommon for diabetic patients to be off a factor of 2 or 3 from the desirable euglycemic target of 100 mg/dL. Poorly managed, the situation can alternate from hyperglycemic to hypoglycemic, or vice versa, in less than an hour.

There are a number of factors that make the delivery of the proper insulin dose difficult:

1) Injected insulin does not impact blood glucose instantly. Even fast acting insulin formulations take hours to be utilized. This means conservative dosing will produce hours of high glucose before supplemental injections can be applied to reduce the glucose concentration. And over dosing can result in hypoglycemia, which presents risk of acute incapacitation or coma.

2) A varied diet requires a concomitant adjustment in insulin dosage. The carbohydrate content of food consumption is rapidly converted to glucose. The correct insulin dose, measured in units, U, necessary for the body to utilize the glucose from the carbohydrate component of a meal, I_{C}, is proportional to the carbohydrate intake, Carbs.

I_{C}=Carbs/CIR (1)

Where CIR, the carbohydrate to insulin sensitivity factor, is particular for each patient and may vary depending upon a patient's condition.

3) When the blood glucose level, BG, is not near a patient's target glucose level, BG_{T}, before a meal begins or at a time after all injected insulin has been utilized, adjustments (in the form of insulin or food depending on the direction of deviation) should be administered to correct for the deviation. The amount of insulin adjustment for high blood glucose deviations, I_{B}, depends on the patient's individual insulin sensitivity factor, ISF.

I_{B}=(BG−BGᵣ)/ISF (2)

I_{B} can be positive if BG is higher than the target or negative if BG is lower than the target. If positive, a dosage of insulin I_{B} should return the patient near to their target blood glucose level. If I_{B} is negative, the current blood glucose (BG) is below the target, so the adjustment would need to involve food ingestion.

4) If I_{B} is negative, food can be consumed to effect an adjustment. Ideally, the amount of food would be just enough to correct the low BG. A food intake sensitivity factor can be used to guide the food intake. Basing the food intake on the food carbohydrate content is currently a preferred method. The recommended carbohydrate intake, Carbs, to correct for a given blood glucose negative deviation, BG−BG_{T} would be

Carbs=−CIR/ISF*(BG−BG_{T}) (3)

−CIR/ISF, is also known as 1/CGR, and can be calculated if one has estimates for their CIR and ISF using either crude estimates or the methods taught in this invention. It is a further benefit of this invention that −ISF/CIR also referred to as CGR (having units of BG mg/dL/gr Carbs), the Carbohydrate to Blood Glucose Ratio, is directly derived from the data diabetic patients routinely obtain.

5) The patient's sensitivity factors can be a function of their condition. So, exercise, stress, illness, etc. can be sources of variation that change how the patient is utilizing insulin. Over longer time periods, the patient's weight and progressing conditions can impact the sensitivity factors, so these should be routinely reevaluated.

The ISF, insulin sensitivity factor is the amount by which an individual patient's blood glucose concentration is reduced for each unit of rapid insulin taken. While it is generally in the range of 30 to 50 mg/dL/U, a more accurate determination is necessary to calculate the part of a bolus insulin injection that is needed to adjust for the blood glucose elevations above a target blood glucose level.

Whether patients are taking episodic blood glucose readings using a glucose strip meter or using a continuous blood glucose monitor, for example, the Paradigm Link™ Blood Glucose Monitor developed by Medtronic and Becton Dickenson, elevated blood glucose levels are commonly encountered by diabetics as a result of inadequate insulin taken for food intake. The ISF value to calculate the insulin dose needs to reflect the patient's response to insulin or there can be a very real risk of inducing hypoglycemia.

Many insulin pumps provide a bolus calculator that utilizes the patient's sensitivity factors. To facilitate access to blood glucose data, some pumps have wireless connectivity to a glucose monitor. Smiths Medical MD, Inc.'s Cozmo pump works with the CozMonitor attached to the back of the pump. The pump receives glucose readings from this attached meter via an infrared communication port. Taking this integration of blood glucose readings to power bolus calculations one step further, the Sooil Development Co. Ltd.'s (Seoul, Korea) DANA Diabecare IISG insulin pump is converged with a blood glucose meter housed within its case.

Determining the patient's CIR and ISF has traditionally been accomplished by applying approximate rules of thumb that generalize a patient's dependence on insulin. The currently practiced method for approximating a patient's CIR and ISF are outlined in the references below. Beginning with these approximate values, corrections or adjustments can be made based on untoward outcomes of applying the estimated sensitivity factors. CIR and ISF are patient specific.

“The 1500 rule is a commonly accepted formula for estimating the drop in a person's blood glucose per unit of fast-acting insulin. This value is referred to as an ‘insulin sensitivity factor’ (ISF) or ‘correction factor.’ To use the 1500 rule, first determine the total daily dose (TDD) of all rapid- and long-acting insulin. Then divide 1500 by the TDD to find the ISF (the number of mg/dL that 1 unit of rapid-acting insulin will lower the blood glucose level) . . . . The 500 rule is a formula for calculating the insulin-to-carbohydrate ratio. To use the 500 rule, divide 500 by the TDD.” Claudia Shwide-Slavin, “Case Study: A Patient With Type 1 Diabetes Who Transitions to Insulin Pump Therapy by Working With an Advanced Practice Dietitian,” Diabetes Spectrum 16:37-40 (2003).

Different constants are proposed in a standard reference book [Diabetes Management in Primary Care, Jeff Unger, Lippincott Williams & Wilkins, 2007, p 485] “To determine the ISF, 1700 is divided by the patient's calculated total daily dose of insulin.”

A concise statement of the current practice is in Practical Management of Type 1 Diabetes, Ira B. Hirsch, Steven V. Edelman, Professional Communications, 2005, SBN 1884735940, 9781884735943, p 103, “The patient's individual correction factor (i.e., the extent to which blood glucose will decrease per unit or rapid-acting insulin based on premeal blood glucose levels) must be determined to adjust prandial insulin doses properly. Although there is no exact method for calculating the correction factor (also referred to as the ‘insulin sensitivity’ factor), many clinicians employ the ‘1800 Rule’ if using rapid-acting insulin (or the 1500 Rule for regular insulin).”

After using one of the above approximation methods, it is currently suggested that patients adjust their sensitivity ratios by isolating either insulin effects or carbohydrate effects. The methods are somewhat demanding and need to be repeated to average out errors. The difficulty in adjusting the initial estimates using these commonly employed methods is that the adjustments require circumstances when only insulin or only carbohydrates are being used to correct blood glucose. These univariate events are awkward to arrange and require encountering circumstances of specific blood glucose values and the opportunity to adhere to the testing regimen. The method to adjust ISF evaluates effects of relatively small insulin doses used to compensate moderate hyperglycemia. For example, when there is high blood glucose, an insulin dose alone may be used correctively. If blood glucose is high by 50 mg/dL a typical correction would be in the range of 1 U of insulin, compared to typical meal insulin dosages in the 5-10 U range. Small doses in the range of 1 U are fairly inaccurate if delivered by syringe. In another case, if blood glucose is low by 50 mg/dL, a carbohydrate intake in the range of 10 grams of carbohydrates might be used, compared to a normal meal consumption of 30-80 grams of carbohydrate. For evaluation of one's CGR, a normal meal must be put off a few hours while the effects of this small intake are evaluated without the interference of insulin.

Here is the ISF adjustment method recommended at a web site for insulin pump users (The Insulin Pumpers Organization):
“BG/I test procedure: Measure your BG/I ratio by checking your body's response to a bolus. If you are comfortable with a one-unit bolus when at 150 then the following procedure will give you a good idea of the blood sugar drop caused by a unit of insulin. The one unit bolus is intended to move your blood sugar levels down by 60 to 75 points. Use a smaller or larger bolus to achieve this target range and calculate the BG/I ratio after completing the test period. CONSULT your health care advisors. If you are uncertain about this procedure, do not proceed . . . . Fast for 4-5 hours prior to beginning the test.
With blood sugar near 150, bolus 1 unit of Humalog. You may adjust your blood sugar using glucose tablets, however wait at least 20 minutes after taking glucose and test your blood sugar again before administering the 1 unit bolus.
Wait 2½ to 3 hours and check your blood sugar, record the difference from the original reading, this is the Insulin to Blood Sugar ratio.
This test could be performed using Regular insulin, however, the wait period would be 4½ to 5 hours rather than 2-3 with Humalog.”

To measure one's CGR the Insulin Pumpers Organization recommends the following method:

“BG/Carb Test Procedure
Measure your BG/Carb ratio by checking your body's response to the ingestion of 4 to 10 grams of carbohydrate in the form of glucose tablets. If you anticipate your blood sugar rise to be less than 25 points, then use two glucose tablets instead of one.
Fast for 4-5 hours prior to beginning the test.
With blood sugar between 80 and 100, eat one or two glucose tablets.
Wait 20 to 30 minutes and check your blood sugar, record the difference from the original reading.
Divide the difference in the blood sugar readings from the beginning to the end of the test by the number of grams of carbohydrate ingested in the glucose tablets. This is the Blood Sugar to Carbohydrate ratio.”

Each of these sensitivity factor procedures are intended to improve on the very general “rules-of-thumb” methods of sensitivity factor approximation by making taking into account actual BG readings on the patient. The chief problems with these methods are: 1) they require that the patient adhere to a special test procedure and in the case of the ISF test, fast at least seven hours from their last meal and 2) the methods use the outcome of a single experiment without regard to the contribution of random noise in the data used or the impact of uncontrolled variables.

The combination of using relatively small stimuli (insulin dose or food amount) and infrequent data gathering occasions results in sensitivity factor calculations that lack precision and statistical power. Generally, there is no attempt to collect a sufficient multiplicity of such determinations and apply statistics to determine a confidence limit for the average sensitivity factor.

Prior art teaches that the ISF and CIR sensitivity factors are to be used by patients to calculate their bolus insulin dosage. This dosage measured in insulin units is the sum of the insulin to compensate for food intake (I_{C}=carbohydrate grams/CIR) plus an insulin correction (either positive or negative) to correct for the deviation from target blood glucose (I_{B}=(BG−BG_{T}/ISF)). (See Equation 8 below.) Examples of the prior art documenting the importance of using the sensitivity factors include: (1) “Continuous Subcutaneous Insulin Infusion Therapy for Children and Adolescents: An Option for Routine Diabetes Care,” Pediatrics, Vol. 107 No. 2 (February 2001), pp. 351-356: “patients . . . were taught dietary strategies to calculate insulin bolus dosing based on insulin to carbohydrate ratio . . . ”; (2) “Using Carbohydrate Counting in Diabetes Clinical Practice,” J. Am. Diet. Assoc.; 98(8) (August 1998) pp. 897-905: “The concept of carbohydrate counting has been around since the 1920s . . . designed to teach clients . . . who are using multiple daily [insulin] injections or insulin infusion pumps how to match short-acting insulin to carbohydrate [intake] using carbohydrate-to-insulin ratios.”

Furthermore, prior art teaches that the patient is to use successive blood glucose readings to determine a change in blood glucose from a known stimulus in order to determine their sensitivity values. Specifically, the change in blood glucose values when only insulin is used (without food intake) is used to calculate a corrected ISF and a change in blood glucose values when only a known carbohydrate intake has occurred (without insulin administration) is used to calculate a corrected CIR. Sometimes, these are performed retroactively when conditions allow a simple sensitivity factor calculation. The standard deviation σ for repeated readings of a single blood sample is about ±5 U or about 5% accuracy (optimistically), the standard deviation of the difference of two readings is √2 σ or about ±8 U. If the BG difference is a much as 20 U to 50 U, a single difference determination will have standard deviations of 16% to 40%, so the sensitivity factors so determined will have these same undesirable low accuracies.

United States Patent Publication 2005/0192494 A1 (“Ginsberg”) discusses iterative fitting of patient data using successive approximations for CIR and ISF beginning with the current values of these parameters. The method involves using an initial ISF and CIR to find better values of these parameters. Ginsberg discloses calculating a plurality of ISF factors for a plurality of days based on the “correct insulin amount” being based on the previous estimate of the ISF and calculating the average.

U.S. Pat. No. 6,544,212 (“Galley et al.”) is a method to inform patients of insulin dosage that utilizes “data from the subject on insulin sensitivity” but does not determine either the insulin sensitivity factor, or the insulin to carbohydrate ratio.

U.S. Pat. No. 7,204,823 (“Estes et al.”) describes an apparatus to manage insulin delivery based on a patient profile which includes “settings . . . selected from the group including target blood glucose, carbohydrate ratio and insulin sensitivity . . . ” There is no discussion or teaching of how the ratio or sensitivity are to be determined or refined.

U.S. Pat. No. 6,691,043 (“Ribeiro”) uses the standard insulin dose calculation (Equation 3) long known in the diabetic literature and teaches a way to fit a polynomial curve to a series of corrected carbohydrate ratios as a function of time of day to provide a CIR profile for the patient. A so-called corrected CIR is calculated (using notation more in line with the notation used herein) by the equation:

CIRc=Carbs/(((BG₂−BG_{T})/ISF+(Carbs/CIR₀)) (4)

Where CIR₀ is the CIR used for the meal, BG₂ is the blood glucose measured after the meal is digested, BG_{T} is the target blood glucose.

In Ribeiro, Equation (4) is applied for each of the meal events of the day. Presumable, if this is not getting the patient into the blood glucose target range, another CIRc can be calculated, but this inevitably leads to patient frustration and can even lead to the patient changing CIR after they have the correct value because deviations will continue to occur. The meaning of the equation is that the correct CIR, CIRc, is the carbohydrate intake divided by the correct amount of insulin needed to get to the target. This is the amount of insulin used for the carbohydrate intake of the meal plus the amount of insulin needed to move BG₂ to BG_{T}. This assumes the amount of insulin used at the time of the meal to correct for the blood glucose deviation was exactly correct. The only source of error is assumed to be due to the CIR being incorrect for that meal of the day. Ribeiro teaches ISF should be fixed by the “rule of 1800” discussed above, and correct future dose calculations can be based on a time sensitive CIR profile determined by the data from a few meals.

Ribeiro teaches CIR can be found using the deviation from BG_{T} for a single meal. Ribeiro teaches a more general correction to CIR in that the blood glucose results of an event involving both food and BG correction can be used to correct the CIR sensitivity factor. However, this method ignores the profound effects of the noise contained within data on sensitivity factor calculations based on a single event. It does not provide a statistically valid method of using a collection of events to easily derive CIR and the other sensitivity factors, ISF and CGR.

Another approach to the problem of adjusting patient sensitivity factors was the use of a causal probabilistic networking model to estimate patient sensitivity factors. [Implementation of a learning system for multiple observations in a Diabetes Advisory System based on causal probabilistic networks, O. K. Hejlesen et. al, in Artificial Intelligence in Medicine: Proceedings of the 4th Conference on Artificial Intelligence in Medicine Europe, 3-6 Oct. 1993, Munich, S. Andreassen, R. Engelbrecht, J. Wyatt, IOS Press, 1993, ISBN 905199141X, 9789051991413, p 67.

### SUMMARY OF THE INVENTION

The present invention provides methods, apparatus and other means to generate the diabetic sensitivity factors from a patient's record of blood glucose, insulin doses administered, and food intake, preferably in the form of grams of carbohydrate intake.

Given the empirical data of an initial blood glucose reading, the food consumption value, and the insulin administered, the resulting blood glucose reading, after these have had time to take effect, is the result of the balance of these factors as influenced by the patient's sensitivity factors and sources of variability. The invention includes a way to find what the operational sensitivity factors are, even when the data largely involves complex events, that is, involving intake of both food and insulin.

The invention further tests the model for adequate data fit and allows calculation of confidence limits for the sensitivity factors as well as probabilities of desirable outcomes of blood glucose management, to set reasonable expectations. Poor data fit reflects either the quality of the data or variability factors of the patient's lifestyle that can be taken into account once identified to segregate sensitivity factors for different lifestyle influences. For example, the calculations can be segregated for exercise days, or work days, etc.

Embodiments of the invention include incorporation of the novel method to find patient sensitivity factors into apparatus including insulin pumps, blood glucose meters, support internet sites, and computer programs.

Another embodiment of the invention enables devices to collect the basic data needed to conduct the derivation of sensitivity factors from other devices where the data resides. The collection can be achieved by cable or wireless transmission. The resulting network can also be used to update an insulin pump with new sensitivity factors for calculation of a recommended bolus dose.

Thus, in one aspect the present invention provides a method of determining at least one diabetic sensitivity factor of an individual based on at least one initial data set, the initial data set. The initial data set includes (1) a first blood glucose reading taken at a first measurement time, (2) a second blood glucose reading taken at a second measurement time following an interval after the first measurement time, (3) the insulin dose administered to the individual during the interval, and (4) a measure of the food intake by the individual during the interval. In this aspect, the method comprises transforming the at least one initial data set to generate at least one transformed data set comprising a pair of transformed variables, the first transformed variable of the pair being the difference between the first blood glucose reading and the second blood glucose reading divided by the food intake measure, and the second transformed variable of the pair being the insulin dose divided by the food intake measure. In this aspect, the method further comprises determining parameters of a functional relationship between the transformed variables and converting said parameters of the functional fit to an estimate of the individual's at least one diabetic sensitivity factor.

Preferably, in this aspect the method further includes obtaining the at least one initial data set. Preferably, in this aspect the second blood glucose reading is taken at a time sufficiently long after both insulin administration and food intake to permit both insulin administration and food intake to affect blood glucose. Preferably, the functional relationship is a linear relationship and said functional fit is a linear fit. Preferably, the parameters of the linear fit are the slope and at least one axis intercept, the value of the slope provides an estimate of the individual's insulin sensitivity factor, and the axis intercepts provide carbohydrate grams per insulin unit as the inverse of the axis intercept of the second transformed variable and blood glucose per carbohydrate grams as the axis intercept of the first transformed variable. Preferably, in this aspect the measure of food intake is grams of carbohydrates contained in the food consumed and impacting the patient's blood glucose level by the time of second blood glucose reading.

In this aspect of the method of the present invention the at least one initial data set can include initial data sets for a plurality of days for an individual who eats a predetermined meal during the interval of each of the initial data sets, the at least one diabetic sensitivity thereby being determined for the predetermined meal.

In the alternative, in this aspect of the method of the present invention the at least one initial data set can include initial data sets for a plurality of days for an individual who undertakes a predetermined activity during the interval of each of the initial data sets, the at least one diabetic sensitivity thereby being determined for the predetermined activity.

In another alternative, in this aspect of the method of the present invention the at least one initial data set can include initial data sets for a plurality of days for an individual experiencing a specific state of health during the interval of each of the initial data sets, the at least one diabetic sensitivity thereby being determined for the specific state of health.

In one embodiment of the method of the present invention, the at least one initial data set comprises initial data sets for a plurality of days and the interval occurs during a predetermined period for each of the initial data sets, the at least one diabetic sensitivity thereby being determined for the predetermined period.

In another embodiment of the method of the present invention, a plurality of initial data sets are obtained, at least one of the initial data sets including an estimated blood glucose reading, and the method further includes omitting data sets including estimated blood glucose readings from the determination of the parameters of the functional relationship.

In yet another embodiment of the method of the present invention, the method further includes testing the initial data sets or pairs of transformed data for reliability and omitting data failing to meet predetermined criteria from the determination of the parameters

In another embodiment of the method of the present invention, the method further includes calculating the range of uncertainty of the at least one diabetic sensitivity factor.

In yet another embodiment of the method of the present invention, the method further includes calculating and communicating the range of blood glucose outcomes that can be expected when using a calculated bolus injection, based on the historic variance of blood glucose outcomes.

In another aspect the present invention provides an apparatus comprising:

(a) memory for storing a database comprising at least initial one data set, the initial data set comprising (1) a first blood glucose reading taken at a first measurement time, (2) a second blood glucose reading taken at a second measurement time following an interval after the first measurement time, (3) the insulin dose administered to the individual during the interval, and (4) a measure of the food intake by the individual during the interval;

(b) means for transforming the at least one initial data set to generate at least one transformed data set comprising a pair of transformed variables, the first transformed variable of the pair being the difference between the first blood glucose reading and the second blood glucose reading divided by the food intake measure, and the second transformed variable of the pair being the insulin dose divided by the food intake measure;

(c) means for determining parameters of a functional relationship between the transformed variables and converting said parameters of the functional fit to an estimate of the individual's at least one diabetic sensitivity factor; and

(d) means for communicating the at least one diabetic sensitivity factor.

In one embodiment, the apparatus according to the present invention further includes an insulin pump for delivering a dose of insulin, and means for calculating the dose of insulin responsive to the estimated at least one diabetic sensitivity factor.

In another embodiment, the apparatus according to the present invention further includes a continuous blood glucose monitor, and means for entering blood glucose readings and the time said readings are taken into the database.

In another aspect, the present invention provides an apparatus comprising:

(a) a data processor for executing a programmed set of instructions;

(b) a memory device accessible to the data processor for storing a database comprising at least initial one data set, the initial data set comprising (1) a first blood glucose reading taken at a first measurement time, (2) a second blood glucose reading taken at a second measurement time following an interval after the first measurement time, (3) the insulin dose administered to the individual during the interval, and (4) a measure of the food intake by the individual during the interval;

(c) a first set of instructions for the data processor for transforming the at least one initial data set to generate at least one transformed data set comprising a pair of transformed variables, the first transformed variable of the pair being the difference between the first blood glucose reading and the second blood glucose reading divided by the food intake measure, and the second transformed variable of the pair being the insulin dose divided by the food intake measure;

(d) a second set of instructions for the data processor for determining parameters of a functional relationship between the transformed variables and converting said parameters of the functional fit to an estimate of the individual's at least one diabetic sensitivity factor; and

(e) an input/output device for communicating the at least one diabetic sensitivity factor.

In one embodiment, this apparatus further includes an insulin pump for delivering a dose of insulin, and a set of instructions for calculating the dose of insulin responsive to the estimated at least one diabetic sensitivity factor.

In another embodiment, this apparatus further includes a continuous blood glucose monitor, and a set of instructions for the processor for entering blood glucose readings and the time said reading are taken into the database.

### BRIEF DESCRIPTION OF THE DRAWINGS

FIG. 1 is a block diagram of an apparatus to calculate diabetic sensitivity factors according to the present invention.

FIG. 2 is an overall flow diagram of a method according to the present invention for the software program component of the apparatus of FIG. 1 to calculate sensitivity factors for a diabetic patient.

FIG. 3 is a detailed flow diagram of the Assemble Events Data step and the Test and Mark Events for Usability step of the method of FIG. 2.

FIG. 4 is a detailed flow diagram of the Generate Transformed Event Parameters step of the method of FIG. 2.

FIG. 5 is a detailed flow diagram for the Find the Best Linear Fit to the Collection of Event Data step of the method of FIG. 2.

FIG. 6 is a block diagram of an apparatus to calculate diabetic sensitivity factors according to the present invention.

FIG. 7 is a functional block diagram of an insulin pump that calculates diabetic sensitivity factors according to the present invention.

FIG. 8 is a functional block diagram of a blood glucose meter that calculates diabetic sensitivity factors according to the present invention.

FIG. 9 is a flow diagram for a method according to the present invention for calculating and using sensitivity factors employing an insulin pump.

FIG. 10 is a flow diagram for a method according to the present invention for calculating and using sensitivity factors employing an insulin pump system utilizing continuous blood glucose monitoring.

FIG. 11 is a high level flow diagram of a local network to communicate data to a device for the calculation of diabetic sensitivity factors according to the present invention.

FIG. 12 is a flow diagram of a method for an apparatus to calculate diabetic sensitivity factors according to the present invention employing sending or receiving data using short-range wireless connectivity.

FIG. 13 is a collection of illustrations showing components of a spreadsheet embodiment of the present invention.

FIG. 14 is a graph showing the insulin-on-board function for rapid insulin according to the present invention.

### DETAILED DESCRIPTION OF THE PREFERRED EMBODIMENTS

The present invention provides a way to solve for sensitivity factors from a patient's routine data. A generalized relationship exists between the blood glucose outcomes, as dependent variables, and the independent drivers of these outcomes. Beginning from an initial blood glucose level, food consumption will increase blood glucose (carbohydrates in the food will impact blood glucose over the short term) and insulin will lower blood glucose. Activity will lower blood glucose, as well. The specific proportionality coefficients to relate the effect of carbohydrates or insulin or exercise to the effects on blood glucose are patient specific, being related to individual size and metabolism, and are called sensitivity factors. Other physiological variables that can impact blood glucose or modulate sensitivity factors include, but are not limited to, time of day, day of the week, stress, and illness. Given the availability of the recording of independent variables of insulin and carbohydrate intake in a patient's daily log or other recording means, their effect on blood glucose can be used to find the corresponding sensitivity ratios that are operating to produce the dependent variable, changes to blood glucose levels. Physiological influences can be studied as independent effects on blood glucose or as modifiers of the sensitivity factors.

Sensitivity factors are needed to calculate a) the insulin dose a diabetic patient would take to treat carbohydrate intake, using CIR (Eq. 1), b) the insulin dose a diabetic patient would take to treat blood glucose deviations, using ISF (Eq. 2), and c) the amount of food to eat to treat hypoglycemia, using CGR (Eq. 3). To find these sensitivity factors, the method of the present invention treats them as unknown parameters of the outcomes relationship. The method uses input data comprising a) blood glucose reading (BG) pre-meal and/or pre-insulin delivery, b) blood glucose reading (BG₂) taken at least an hour after a meal where no insulin has been taken or at least three or four hours after when insulin has been taken, c) the grams of carbohydrate contained in food ingested (Carbs), and d) the actual units of all insulin (1) affecting the period between taking of blood glucose (BG) and BG₂. Iₐ is the dose of insulin taken with a meal or more accurately includes corrections for the portion of insulin taken close to the time of either BG reading, see discussion concerning Equation 45 below.

The carbohydrate intake variable, c) above, is relatively easy to obtain since the mandate in the United States and some other countries for printing the nutritional content of food on food packaging. The carbohydrate content is usually provided for a “serving” of the food. Food database information including the serving size and carbohydrate content can be used to estimate the carbohydrate content of a food item. The carbohydrate content for thousands of food items is available in the form of nutritional databases such as the USDA National Nutrient Database for Standard Reference, [Handbook of Nutrition and Food, Carolyn D. Berdanier, CRC Publishers, 2001, p 564].

The patient's carbohydrate intake for a given food item is the product of the carbohydrate content per serving and the number of servings consumed. The number of servings consumed is the ratio of either the weight of the food item consumed to the weight of a standard serving, or the ratio of the volume of the food item consumed to the volume of a standard serving. This can also be provided by an apparatus that weighs or otherwise measures food quantity and automatically provides the user with the product of the amount of food and the specific carbohydrate content, as disclosed in United States Patent Application Publication 2006/0036395 A1, incorporated herein by reference.

Given food consumption, insulin delivery, and starting blood glucose values, the true biological sensitivity factors result in the patient's actual subsequent blood glucose reading, BG₂. The values of the sensitivity factors used by a patient for their bolus calculations are estimates of the true biological sensitivity factors operating at that time. This invention surprisingly provides the true biological ISF, CIR, and CGR derived from the patient's data involving general use of insulin in combination with the effects of food consumption. No special treatment routines are required and the method can utilize the entirety of the patient's record rather than attempting adjustments based on single event data. Incomplete or inaccurate data records are not a useful part of the record used for sensitivity factor derivation.

The sensitivity factors that can be determined using this method are intended for use by the patient going forward to better manage their blood glucose levels. The sensitivity factors are not necessarily assumed to be constants for the patient. The method teaches ways to determine sensitivity factor dependence on factors such as time of day, or particular kinds of days, such as work days, exercise days, or sick days.

The ISF, CIR, and CGR determined with this method from the patient's recorded data is then an appropriate value to use for five general situations:
1. To determine an insulin dose component, I_{B}, to correct high blood glucose (BG) deviations (hyperglycemia) observed at any time using the determined ISF:

I_{B}=BG_{Δ}/ISF (5)
where BG_{Δ}=BG−BG_{, and BG}_{T} is the blood glucose target for the patient.
2. To determine in insulin dose, I_{C}, to take to allow neutralization of the glucose elevated by food consumption where Carbs is the carbohydrate content in grams of the food. Equation (4) uses the determined CIR, the best value for an individual's recommended carbohydrate to insulin ratio.

I_{C}=Carbs/CIR (6)
3. To determine the correct food intake to treat hypoglycemia without overshooting or undershooting the patient's blood glucose target.

Carbs=(BG_{T}−BG)/CGR (7)
4. To determine the insulin dose when there is a need to correct for blood glucose deviation and food consumption. Then, the recommended insulin dosage, Iᵣ, will be:

Iᵣ=I_{C}+I_{B}

Iᵣ=(Carbs/CIR)+(BG_{Δ}/ISF) (8)
5. To determine the insulin dose where there are other factors, F, affecting BG and SF, is the corresponding sensitivity factor for that factor determined by application of the methods provided by this invention.
Then, the recommended insulin dosage will be:

Iᵣ=I_{B}+I_{C}+I_{F}_{ᵢ} (9)

Iᵣ=(Carbs/CIR)+(BG_{Δ}/ISF)+ΣFᵢSFᵢ (10)

To find values for ISF and CIR we use a series of actual patient data. It does not matter if the patient has been using the best dosing or even any consistent dosing approach. In other words, it is not a requirement of this method that the patient use any prior sensitivity values. We do, however, need the patient to take blood glucose (BG) readings before each meal, record the actual insulin dosage taken before the next blood glucose reading, and record their calculation of total carbohydrate intake at each meal. The quality of the data will, of course, affect the quality of the derived sensitivity factors. For example, if a lot of food consumption is inaccurately estimated, this will introduce noise. If a lot of food is simply not recorded, this introduces a bias and the sensitivity factors will appear smaller than true. One way to improve the method is for the patient to mark less reliable data where, for instance, carbohydrate intake is an estimate or guess. This data could be eliminated from consideration so that sensitivity parameters are based on only the more accurate data.

At the heart of the new method is the record of actual insulin doses the patient has taken. Iₐ is the actual amount of insulin a patient takes to treat the effects of high blood glucose and/or the anticipated effects of ingesting food. It may be injected by syringe or infused from an insulin pump. If inhaled insulin is used, a best estimate of the insulin delivered systemically must be recorded. It may involve a single dose or multiple doses taken between the blood glucose (BG) values used before the meal and sometime before the next meal. Iₐ does not include the insulin delivered by basal programs of an insulin pump or by periodic injection of long-term forms of insulin, such as Lantus. The basal insulin dosage is calibrated to keep blood glucose concentration level in the absence of food intake.

The model assumes the change in blood glucose concentration observed before the next meal, BG−BG₂, where BG is a reading taken before the current meal and BG₂ is a reading taken before the next meal, is due to the difference between the actual insulin taken, Iₐ, and the amount that is required to balance the carbohydrate intake, Carbs, corrected by the ISF sensitivity factor. The excess insulin amount is just Iₐ−Carbs/CIR. So,

Iₐ−Carbs/CIR=(BG−BG₂)/ISF (11)

If Iₐ is greater than Carbs/CIR, BG₂, read three or four hours later or at the start of the next meal, will be less than blood glucose (BG) read before the start of the current meal. As an example, if Iₐ−Carbs/CIR is 0.5 U and the patient's ISF is 50 mg/dL/U then BG−BG₂ would be 25 units, meaning a BG drop of 25 mg/dL would be observed. As each BG reading includes noise in the range of ±10 units, the change of BG from a single determination can be somewhat inaccurate and therefore misleading. However, fitting a number of events, where the number is at least 10 and preferably greater than 30, reduces the error by averaging out the random error of individual measures. The events must be chosen without bias.

Rearranging 11, we can obtain the Equations 12 or 13.

Iₐ/Carbs=1/CIR+(1/ISF)(BG−BG₂)/Carbs (12)

(BG−BG₂)/Carbs=ISF*Iₐ/Carbs−ISF/CIR (13)

It is customary to plot a dependent variable for the y-axis. Respecting this convention, the change in blood glucose (BG−BG₂)/Carbs of Eq. 13 is more logical as the dependent variable then Iₐ/Carbs of Eq. 12, as BG₂ comes about hours after the Carbs and Iₐ are applied to the patient. If we use this convention to generate a plot, plotting (BG−BG₂)/Carbs data as the y-coordinate against Iₐ/Carbs as the x-coordinate should display a correlation between the y and x values according to Equation (13). A best-fit linear relationship gives ISF as the slope of the data (dy/dx) and −ISF/CIR for the y-intercept. The y-intercept, −ISF/CIR is the reciprocal of the carbohydrate to blood glucose ratio, CGR, used to correct hypoglycemia. (1/CIR is easily obtained in this representation of the data as the x-intercept.)

Continuing to record carbohydrate intake at each meal, insulin delivered, and blood glucose readings (BG) before meals and three to four hours after meals (BG₂) and using Equation (13) provides ongoing best fits of the sensitivity factors to explain the changes observed in BG. Data can easily be segmented by meal type to permit more precise recommendations for each meal of the day. Similarly data can be segmented for part of the week, sports participation, illness, etc. to see if the best-fit sensitivity factors are significantly affected by these segmentations.

The span of time covering the dataset used in the analysis involves a trade-off. Using longer intervals, for example, more than a year, provides more data points for the analysis providing more degrees of freedom to reduce statistical uncertainty. On the other hand, the sensitivity factors can themselves be a function of changes in patient health, weight, or circumstances. We would like the dataset to reflect current conditions rather than historical periods that may no longer be representative of the patient's current diabetic condition. If a patient is conscientious about recording their input data, a month of data contains about 100 data points corresponding to the number of meal intervals or events. This is adequate for aggregate and even segmented analyses limited to a specific meal of the day. Missing data occurs if any of the four input variables characterizing an event is missing or largely uncertain, for any reason. The regression analysis is performed only on available data points so explicitly missing data introduces no systematic error. Missing data contributes to taking a longer time to build a robust linear relationship. The analysis can use events with food and insulin or with only food intake; however, it does not include those events where only insulin is used to correct hyperglycemia, as the transformed variables require division by the food intake value.

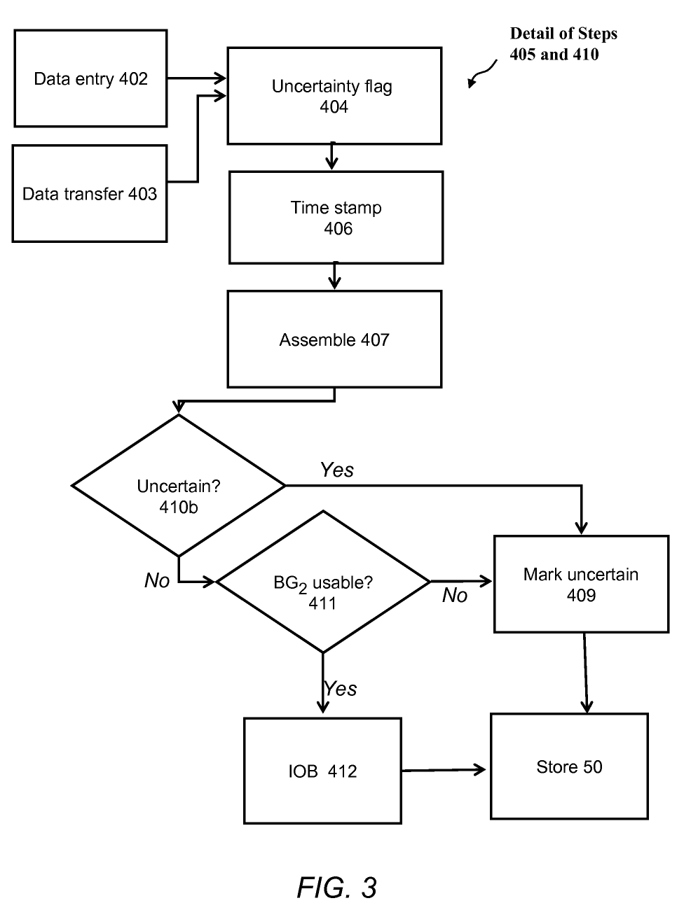

Individual data points can vary in their reliability. While we would hope to be using only reliable input data in this method, not all data obtained by a patient is equally reliable. Relatively unreliable data should ideally not be used for the analyses of this invention. To facilitate elimination of unreliable data requires methods to so designate specific input data. This is considered below and a flow chart for data acceptance on the basis of reliability can be developed (FIG. 3).

Insulin values are unreliable if the patient is not sure about the amount of insulin administered. If the patient forgot to enter the value at the time of administration or for any reason has to guess regarding this value, they should mark the data entry as uncertain.

If the program does not include means to correct insulin doses to working insulin as in Eq. 45, insulin values are unreliable if the time between insulin delivery and the second blood glucose (BG)) reading is less than the time it takes for the insulin to work. In this case, there is still insulin in the body that has not acted on the patient's BG level. This can be detected by program steps that check time intervals and automatically mark some lₐ as uncertain. The time for insulin to work varies for each type of insulin in the market:
Rapid acting insulin, for example Novolog, is generally used by the body by four hours.
Short-acting insulin (also called regular insulin) action ends in about 5 to 7 hours.
Lente insulin and NPH insulin function up to 18 to 28 hours.
   [Principles of Insulin Therapy, Cheng and Zinman, in Joslin's Diabetes Mellitus, ed. Joslin, Kahn, Weir, King, Jacobson, Moses, and Smith, Lippincott Williams & Wilkins, 2004, p. 661]

Blood glucose readings are unreliable if the patient is unsure about the value they are entering and does not check with the BG meter memory as an aid to data entry. In this case, the patient will mark the event as containing uncertain data. Ideally, all blood glucose (BG) readings would be downloaded from the patient's meter to the database used for the analysis of sensitivity factors. This is easiest if the database is within the BG meter or the database can be filled by a direct connection to the BG meter through cable or wireless communications.

The most common source of unreliable input data is in regard to food intake, and in particular carbohydrate intake. Two factors affect the accuracy of the carbohydrate intake: specific nutritional content and portion size. The specific nutritional content of a food is available either on the label of the food, in a nutritional content database, or by calculation for a recipe, or by approximation to a similar food, or by approximation to a similar meal. In the first two sources, the patient has found nutritional content information pertaining to the exact food being consumed. The grams of carbohydrate for a serving is printed on the label, where a serving is defined as a volume, e.g. ½ cup, or weight, e.g. 67 gr. or 4 oz., or a standard amount, e.g., medium sized peach. This is fairly accurate information. However, this content per “serving” needs to be multiplied by the number of “servings” in the actual portion consumed. Much inaccuracy is introduced in poorly estimated portion sizes. The only way to overcome this source of error is to weigh or measure one's portion as accurately as possible. Whether estimated or measured, the value recorded may not include additional servings or subtract left over portions. Unless care is taken to consume what is accurately measured, the portion size may easily be inaccurate by a factor of two. Of course many things we eat are not packaged with nutritional labels. So, home recipes are not exactly the same as anything in a food database, though a reasonable nutritional value per ounce can be estimated. Or a plate of food in a restaurant is estimated to have 90 grams of carbohydrate because it is more food than a familiar meal known to be 70 grams of carbohydrates. The patient needs to be asked two questions regarding a carbohydrate intake value: 1) Do you know the carbohydrate content for this exact food? And 2) Did you measure the amount of the food you ate and calculate the carbohydrate content? If either of these is answered in the negative, the Carbs value should be considered uncertain and the event containing it should be marked as uncertain and not be used in the sensitivity factor derivation.

Often snacks are consumed between meals or as part of a meal and are not entered to the database and are not provided compensating insulin. The resulting higher than expected blood glucose (BG) readings introduces bias to the model. Ideally, an event that includes unaccounted food consumption would be marked as uncertain by the patient to allow its exclusion.

If exercise is a factor used in calculating sensitivity values, the amount of exercise needs to be input. While there are some devices that provide quantitative caloric expenditure based on heat dissipation, and some athletes take the trouble to use motion monitors such as a pedometer or energy expenditure readout on an exercise device, most exercise is ad lib and represents different demands on the patient each time. Usually exercise is entered in binary form for a day. That is, “Yes” or “No.” Sometimes the time of exercise can be an additional factor for analytical purposes. Various approaches can be implemented to test the sensitivity factors for a dependence on exercise. The database can be segmented by exercise metric and time after the exercise event to see if a reliable sensitivity factor can be derived.

With a graphical plot of the data, with training, a best-fit line can be drawn and the slope and intercepts read off the graph.

It is not necessary to utilize a graphical plot to derive the slope and intercept of the best linear fit to the data. Linear regression equations can be programmed to operate using the dataset. Given a set of data, with n data points, the slope (m), y-intercept (b) and correlation coefficient (r), a measure of quality of the dataset's fit to the model, can be determined using the following standard equations:

$$\begin{matrix}m = ( n \sum ( xy ) - \sum x \sum y ) / ( n \sum {x}^{2} - {( \sum x )}^{2} ) & ( 14 ) \\ b = ( \sum y - m \sum x ) / n & ( 15 ) \\ r = ( n \sum xy - \sum x \sum y ) / \sqrt{( \begin{matrix}n \sum {x}^{2} - \\ {( \sum x )}^{2}\end{matrix} ) ( n \sum {y}^{2} - {( \sum y )}^{2} )} & ( 16 )\end{matrix}$$

(Note that the limits of the summation, which are 1 to n, where n is the number of events include in the dataset being used to find a linear relationship, and the summation indices for the x and y values of the events, have been omitted from the notation.)

Similarly, the confidence interval of the slope and intercept are readily available statistical functions. The standard error of the slope is

SE=\sqrt{Σ(yᵢ−ŷᵢ)²/(n−2)}/\sqrt{Σ(xᵢ− x̅²)} (17)

where yᵢ is the value of the dependent variable for observation i, ŷᵢ is the estimated value of the dependent variable for observation i, x, is the observed value of the independent variable for observation i, x̅ is the mean of the independent variable, and n is the number of observations. [Standard Statistical Calculations, Moore, Shirley, and Edwards, Wiley, 1972, p 58]. The confidence interval for the slope is the slope±(SE times the critical value). The critical value is a number based on a t-score with n−2 degrees of freedom and a defined probability for the confidence range. The critical value is roughly 1.3 for a 0.90 probability with n from 30 to 300, meaning the 90% confidence limits are the calculated best estimate slope±1.3*SE.

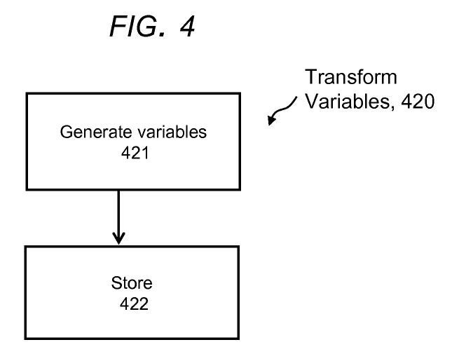

If a patient records BG, Carbs, Iₐ, and BG₂ regularly, a spreadsheet program can easily generate graphs of the transformed variables (BG−BG₂)/Carbs versus Iₐ/Carbs and the statistical fit for ISF and CIR as well as the uncertainty of these generated parameters for any confidence level. FIG. 4 describes the generation of the transformed variables used for the analysis.

The parameters of the fit line can be produced within any device with computational capability where the patient inputs BG, Carbs, and Iₐ, or where the device has knowledge of these variables collected through any variety of data acquisition or communication means. The time associated with each data value is important to assemble the appropriate event dataset and to combine the BG values to produce BG−BG₂. The device can then calculate CIR and ISF by a linear fit to Equation (13).

We want to communicate to the patient that the calculated sensitivity parameters may not be known precisely because a) the data is not perfectly described by the model indicated by a low r and b) there is not enough data to have a precise definition of their response model, indicated by high parameter standard errors. The correlation coefficient, r, describes the fit to a linear model. If this is poor, the patient is encouraged to reduce noise that may be in the data by being more conscientious in data generation and marking uncertain data. By the time n is 30 or more, there should be enough events to define the sensitivity factors adequately. Although parameters of fit to a noisy dataset can be made arbitrarily precise, as n grows large, we want to encourage higher r-values so the model is representative of the data. The absolute value of the correlation coefficient, r, should be 0.7 or greater. (|r|>0.7)

Separately, it should be communicated to patients that the outcome of using the model to calculate bolus dosages will result in a range around their target BG_{T} because outcomes (BG₂) include significant random sources of variation that remain even when the sensitivity factors have been well characterized.

To this end, the present invention provides for calculation of confidence limits on the derived sensitivity factors and a calculation of standard deviation of the data about the model. Patients are provided with measures of the uncertainty of their sensitivity factors and measures of their outcome, BG₂, variability that will help them deal more rationally with their diabetes management. At the same time, educators and doctors will begin to collect experience with database sensitivity factors and the statistical measures of model agreement. This new information can direct educators to help patients reduce their sources of variability.

By the method of the present invention, sensitivity factors can be obtained from a data set by the plotting of data. If a diabetic patient has an interest in achieving tighter control of their blood glucose excursions, they are today counseled to determine and adjust their sensitivity factors and use these to gauge insulin dosages with meals and to correct high BG, as well as to more accurately treat hypoglycemia with food. To accomplish this, patients are trained to record their insulin doses, carbohydrate intake, and blood glucose readings. However, there are currently no methods to allow the patient to extract sensitivity values from complex data where food and insulin are involved and there are no methods to allow them to use a set of data to avoid being misguided by fluctuations inherent to single events.

Beginning with a time-ordered list of data on a patient's BG, actual insulin doses (Iₐ), and meal carbohydrate intake (Carbs), with the aid of only a calculator, it is not difficult to create two new lists of transformed variables Iₐ/Carbs and (BG−BG₂)/Carbs, where these variables have been defined above. Using graph paper, the x-axis can be set to range from 0 to the highest value of Iₐ/Carbs and the y-axis can be set to cover the range of (BG−BG₂)/Carbs in the list. A data point is plotted for data where both of the new transformed variables are known for a specific time interval defining an event. In other words, the blood glucose (BG) values before a meal and the resulting BG some hours after the meal, the insulin dose at the meal, and the carbohydrate consumption early in the time interval are all necessary to generate a plotted data point. Sometimes, either of the transformed variables may be zero, but the event should be excluded whenever Carbs is zero.

If the collection of data points appears to display a linear correlation, a line can be drawn providing a reasonable fit to the data and extended to intercept both axes. The negative of the slope, dy/dx, of the line is the ISF. Where the line crosses the x-axis is the inverse of the carbohydrate to insulin ratio, CIR, and where the line crosses the y-axis is the blood glucose to carbohydrate ratio, CGR.

From this embodiment and the method in general it can be seen: a) that this method requires more than two events of sufficient confidence to produce data points in order to generate a line and a sense of the noise attending to the method (preferably more than 10 events should be plotted); b) that the data points include meals where both food and insulin can be involved; and c) that the more data points one utilizes, the more accurately the sensitivity parameters can be established.

The data spread around a best-fit line results from a) the variability in patient responses due to additional factors and b) sources of noise entering the calculation of the transformed variables. If no line is apparent, despite proper treatment of the data, there is likely to be an underlying problem which could include: a) the patient is not obtaining data in a timely fashion, b) the patient is not correctly using accurate portion size measurements to calculate correct carbohydrate intake, or c) the patient is eating snacks that are not reflected in the dataset.

Commercial applications of the method described above involving hand plotting of data include pads and instruction materials to facilitate the procedure. Diabetic educators are paid to instruct patients in finding their sensitivity factors. This method allows them to utilize accumulated data rather than hunting for individual events that suggest a sensitivity value and finding a range of such events that inevitably yield a range of answers.

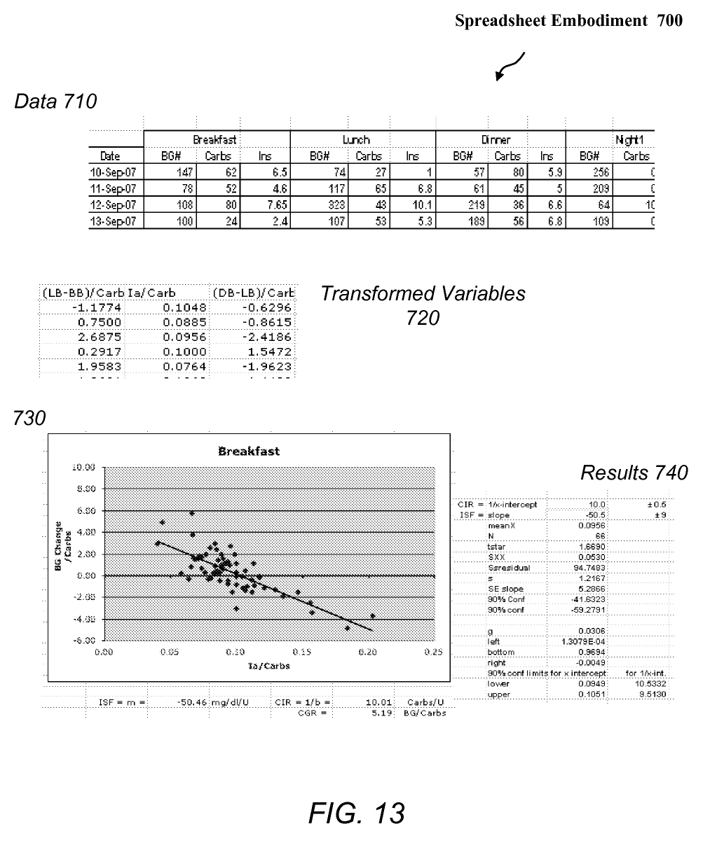

Sensitivity factors can also be obtained from a data set using a spreadsheet program. Data from a hand written log can be manually entered into a properly set up spreadsheet program such as Excel [Microsoft Corporation] as shown in FIG. 13. Similarly, data from a database stored within a blood glucose meter or an insulin pump can be downloaded into a spreadsheet program using the device's data downloading capabilities. The transformed variables, Iₐ/Carbs and (BG−BG₂)/Carbs, introduced by this invention are then automatically calculated. Checking that data conform to rules can be included in the spreadsheet calculations. These rules include proper time intervals between insulin and BG readings. Data not conforming to the rules or that include a patient declared flag for uncertainty can be automatically eliminated. A chart of the included data points can be displayed and the slope and intercept and their confidence intervals can be automatically calculated, as is also displayed in FIG. 13.

Commercial software can be sold to facilitate this method that are stand alone implementations not requiring any general purpose spreadsheet software on the user's computer. These can be used at every meal to facilitate collection of the BG, Iₐ, and Carbs data used for sensitivity factor calculations on all the trailing data. Segmentation of the dataset for meals or days marked for illness, stress, or exercise can be provided. These implementations are made possible by the use of transformed variables to generate a predicted linear relationship based on the sensitivity factors needed to determine insulin and food dosages to correct blood glucose high and low imbalances, respectively.

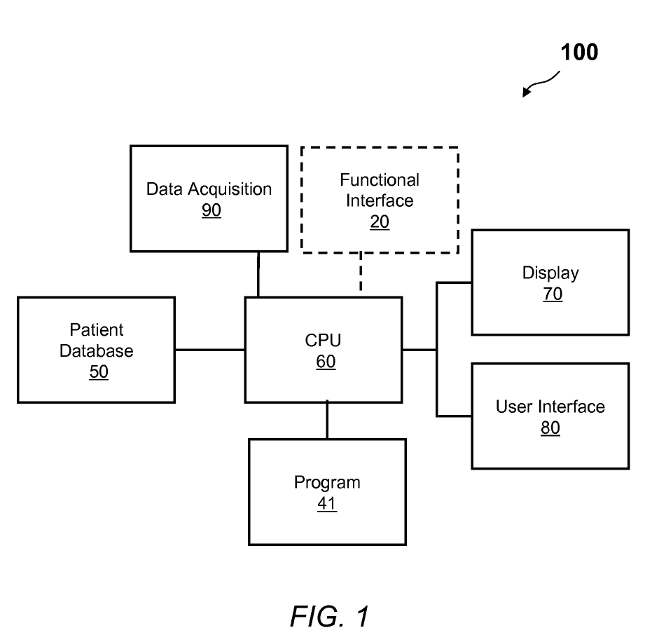

FIG. 1 is a block diagram illustrating the design of an apparatus to calculate diabetic sensitivity factors 100. A central processing unit or CPU 60 is used under control of a program 41 to direct the operations and transfer of data. Data on patient events is acquired by module 90. This is generalized as the data may be obtained by an interface and means to perform other functions 20 of the apparatus 100, by communication with other devices to obtain necessary data at the time of data acquisition, by communication of a data set from another device, or by input of some or all the data by the user, or any combination of these methods of data input to supply the assembly of the requisite dataset. After appropriate filtering of data, assembling of data records, and optionally sorting of events into a sequence, the data is assembled as a patient dataset and stored in memory 50. At a minimum, the dataset requires blood glucose readings, bolus insulin doses, and food intake data (Carbs) that can be grouped into events. The dataset or portions of the dataset are processed by the program 41 to yield the patient's sensitivity factors. The user interface 80 allows the user to enter or correct data and to direct the CPU 60 to display any of representations of the data, a plot of transformed data, the sensitivity factor results, or their confidence limits. These displays appear to the user on a display 70 that is envisioned as an optical interface, but can be design for aural communication when required. An uncertainty flag can be generated for any data entry using a user interface 80 by clicking on a box marked “value is a rough estimate” or an equivalent expression when data are acquired.

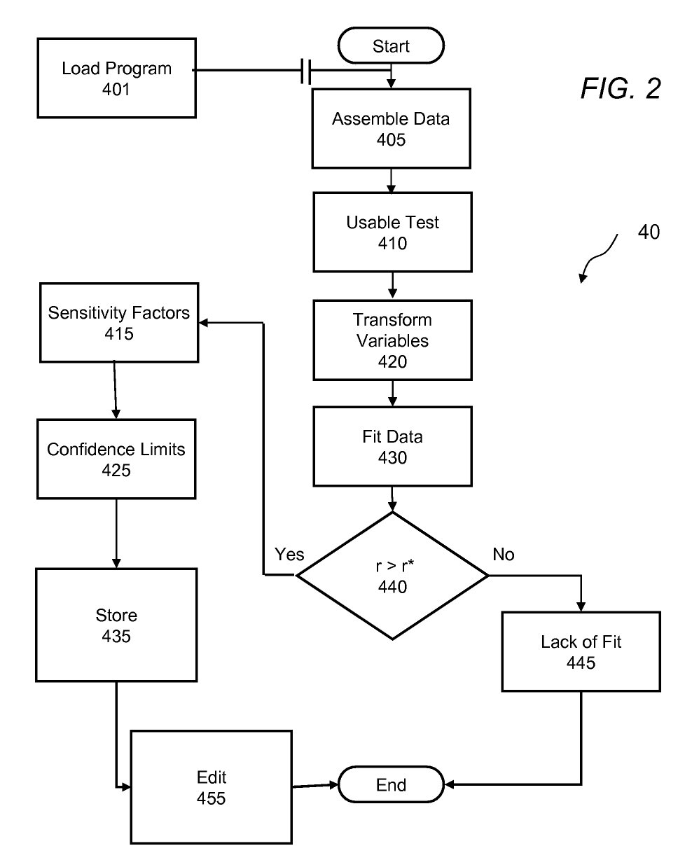

Examples of functions that may be integrated within 20 include means of measuring blood glucose or means of delivering insulin, or means of calculating Carbs for a meal. In each case, the data relating to the function would be stored along with its timestamp. Apparatus 100 would obtain whatever other data is needed (whether BG, Iₐ and/or Carbs) and their timestamp to permit method 40 of FIG. 2 to be carried out. By a timestamp is meant the date and time accurate to the minute for when a data value was established. These values can be imported from another device using a cable or local networks such as Bluetooth or manually input by user interface 80.

Currently, insulin pumps commonly record Iₐ, the bolus insulin delivered to the patient, and the time of the delivery and in many models help the patient to calculate the bolus insulin dose by asking for entry of meal Carbs and pre-meal blood glucose, BG, to generate a bolus insulin recommendation based on the current values of the patient's sensitivity factors. In an embodiment of this invention, an insulin pump is enabled to calculate sensitivity factors using these stored values of BG, Carbs, and Iₐ that have been employed for the currently implemented bolus insulin calculation routines. When these data are properly stored and further processed according to the method of this invention, such as illustrated in the block diagram of FIG. 2, patient sensitivity factors can be derived from the data. These derived patient sensitivity factors can be displayed to the patient for consideration of use of the derived sensitivity factors or the pump may update these parameters automatically.

The present invention also provides apparatus having the ability to calculate sensitivity factors. To produce an apparatus that can calculate and communicate sensitivity factors for a patient requires a basic structure for the apparatus such as shown in FIG. 1. The data must be processed to generate the sensitivity factors based on the mathematic relationship uncovered in Equation 12 or 13. There is latitude in how the linear relationship is calculated. For the examples of embodiments of this invention, the sensitivity factors are calculated according to the outline described in FIG. 2.

Component 41 of FIG. 1 is the program for calculation of sensitivity factors. The method 40 employed by the program 41 is schematically detailed in FIG. 2. For any device to calculate the sensitivity factors according to the principles taught in this invention, they need a) access to a database of time delineated data on pre-meal and post-meal blood glucose readings BG and BG₂, actual insulin delivered (Iₐ), and food intake (preferably Carbs), and b) a processor and computer program set to perform data quality control and mathematical operations on the data to calculate the sensitivity factors based on fitting a linear model of the correlated trans-formed parameters Iₐ/Carbs and (BG−BG₂)/Carbs or their equivalents. In addition, for quality control purposes, the program should be provided the patient's insulin type used for bolus insulin; a lookup table has the time required for this insulin type to complete 90% of its action, TI, used as an event rejection criteria for insulin delivered too soon before blood glucose readings are taken. Alternatively, insulin-on-board adjustments can be made as described below.

The schematic diagram in FIG. 2 illustrates the novel process for finding the sensitivity factors using a method 40 according to the present invention. To accomplish its task the method 40 can be embodied within a computer program either in high-level language or a list of machine code instructions to direct the operation of the CPU 60. The program incorporating the handling of data and fitting of parameters to derive sensitivity factors is prepared to achieve the tasks of method 40 and stored in ROM or loaded for execution into a dynamic memory location of RAM, step 401. The next step 405 required is the assembly of a patient's data describing blood glucose outcomes following an event characterized by use of insulin, consumption of food, or a combination of both. A method according to the present invention for constructing data records is illustrated in the block diagram of FIG. 3. Additional elements that are included in the events data assembly step 405 can include a command to obtain data from an external data source, and selection of subsets of the data to evaluate sensitivity factors specific to the subset, such as certain meals or days of the week.

The event data being assembled in the event data assembly step 405 comprise BG or BG₁, BG₂), all Iₐ, all Carbs, uncertainty flags, and insulin-on-board (IOB₁ and IOB₂) if these are not zero. Here, subscript 1 indicates pre-meal and subscript 2 indicates post-meal event values. All Iₐ and all Carbs sum multiple Iₐ and Carbs occurring in the event interval. For each of the event parameters there is a corresponding timestamp or time of data entry. In a further testing step 408 (not shown), a sub step within the event data assembly step 405, BG₂ is tested for its use as the result of actions of the preceding meal or event. In a data testing step 410, the event itself is evaluated for usability for parameter fitting. If any of the variables BG, BG₂, and both Iₐ and Carbs is missing, the event is marked unusable, step 409. If there are uncertainty flags for any of BG, BG₂, Iₐ, or Carbs, the event is marked unusable. If the time interval between a Carbs or an Iₐ, and a BG reading is smaller than criteria for each type of interval, the event may be marked unusable. If an IOB, insulin-on-board calculated by Eq. 45, is greater than a set percentage of Iₐ, preferably 35%, the event is unusable because IOB is not a very accurate calculation. If the time interval between BG and BG₂ is too small or too large, the event is marked unusable. A general scheme for the testing of events is illustrated in the block diagram of FIG. 3.

The next step in the method 40 is to generate transformed event parameters step 420, further illustrated by the block diagram of FIG. 4. The transformed event parameters are (BG−BG₂)/Carbs and Iₐ/Carbs, necessary for the calculation of sensitivity factors from linear fits of Equations 12 or 13.

In the next step, the linear regression step 430, the program can optionally set segmentation conditions such as meal type, date range, or other conditions and the data points in the transformed variable space are fit by linear regression as by application of Equations 14 and 15. In this linear regression step 430, the appropriate data events are selected, the transformed variables are assigned as either the x or y variable, and sums of x, y, x², y², and xy over the included data events are calculated. These are employed in Equations 14 and 15 to derive the slope and y-intercept of the linear model for the transformed data.

In the following step 440, the correlation coefficient test step, the correlation coefficient r is calculated by Equation 16 and tested for use of the model in sensitivity factor calculations. If the absolute value of the correlation coefficient (|r|) is below a critical value, e.g., 0.7, the patient is informed about the segmentation basis set size and the failure to obtain a good enough linear model. If r is adequate in the correlation coefficient test step 440, the program proceeds to convert the calculated slope m and y-intercept b values to sensitivity factors depending on the form of Equations 12 or 13 employed in the generate sensitivity factors step 415 as shown in FIG. 2.

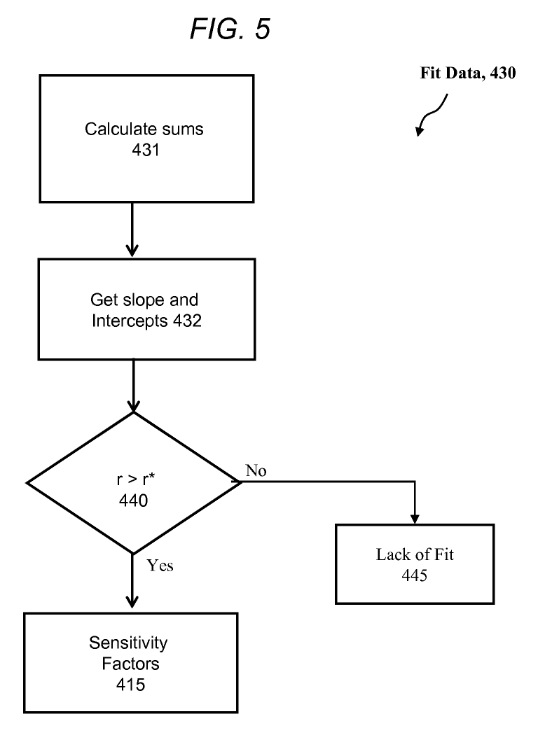

In the next step, the generate confidence limits step 425, confidence limits are derived for the sensitivity factors using standard statistical methods, as illustrated in FIG. 13 and discussed below following discussion of the method illustrated in FIG. 5 to fit the regression line. In the following step shown in FIG. 2, the store and communicate sensitivity factors and confidence limits step 435, the segmentation basis, the number of events included in the model, and three derived sensitivity factors CIR, ISF, and CGR and their confidence limits are communicated to the patient or medical professional.

In the following step, the present sensitivity factors step 455, the patient or medical professional accepts the sensitivity factors that have been calculated or adjusts those values. The adjustments may be chosen to impose more gradual changes to a patient's program or to compensate for differences in past and upcoming conditions. Once adjustments have been made or the values accepted, the sensitivity factors are stored for a variety of uses. These uses include a) determining if one segmentation basis is statistically significantly different from another segmentation basis to warrant use of separate sensitivity factors according to the segmentation basis, and b) storing the results for use in calculating recommended bolus insulin doses.

FIG. 3 is a block diagram schematically depicting additional portions of the present method, including an assemble events data step 405, and a test and mark events for usability step 410, both steps of the method 40 illustrated schematically in FIG. 2. The assemble events data step 405 (FIG. 3) is comprised of multiple substeps 402, 403, 404, 406, and 407. The assemble events data step 405 begins with a data entry substep 402 or a data transfer substep 403 in which at least the necessary data for the calculation of sensitivity factors, at a minimum, BG, Iₐ, and Carbs, is entered or transferred. In addition to the data itself, a timestamp can be entered, transferred, or generated to keep track of the time of data entry. Uncertainty flags are also acquired if the data include these.

In the assign timestamp substep 406, the timestamp for each data value is established either as the time of entry, or by positioning it as originating between the timestamps of other data values recorded before and after the datum entry. For example, an Iₐ value without a timestamp is received after a timestamped (t1) BG value and before a later timestamped (t2) BG value. 406 can assign a timestamp to the Iₐ value as later than but close to t1.

In the next substep, the assemble events substep 407, events are assembled. An event is defined by data acting between BG and the following BG₂. Within this interval there can be administered insulin Iₐ and food intake, Carbs. If there are multiple Iₐ values or multiple Carbs values acquired between the two event-defining BG values, these are summed to a total Iₐ, ΣIₐ_{ᵢ} or a total Carbs, ΣCarbsᵢ for the event. If any components of summed values are marked uncertain, the sum is marked uncertain. IOB corrections may be necessary as discussed below.

If there are any entries for Iₐ or Carbs intake between two BG readings, an event is described by the earlier BG, the Iₐ data and/or the Carbs data occurring between the earlier and the later BG reading, designated BG₂. Each of the values described in the event include a timestamp. Note that each blood glucose (BG) reading can be used as the second BG of one event and again as the initial BG for the following event. Each event has the structure:
BG, BG timestamp, (Uncertainty flag)
Iₐ₁,Iₐ₁ timestamp . . . Iₐₙ,Iₐₙ timestamp, (Uncertainty flag)
ΣIₐᵢ
Carbs₁, Carbs₁ timestamp . . . Carbₙ,Carbₙ timestamp, (Uncertainty flag)
ΣCarbsᵢ
BG₂,BG₂ timestamp, (Uncertainty flag)
Use Flag (set by 410b or 409)

Data acquired from other instruments should all include timestamps for each data element. However, an apparatus may need to deal with data that is not timestamped. In that case, the logical structure of data acquisition of the other instrument can be used to construct timestamps. For example, if an insulin pump stores blood glucose (BG) and Carbs before suggesting an insulin bolus (based on sensitivity factors) and the actual Iₐ delivered is recorded and timestamped, timestamps for the BG and Carbs can be construed to be a few minutes earlier than the associated Iₐ's timestamp.

The substeps 410b, 409, and 411 of FIG. 3 comprise the test and mark events for usability step 410 of FIG. 2. In substep 410b, an event is marked uncertain if any of BG, BG₂, Iₐ, or Carbs is marked uncertain. The uncertainty flag part of an event can be set to 0 meaning “Not for Use.” The test and mark events for usability step 410 comprises a quality control process in which the data within the event are examined and the flag set at 1 if no problems are encountered examining the data.

If there are no Carbs data contained within the event, the event's use flag is set to zero, meaning the event will not be used for the calculations to follow. This avoids the problem of transforming variables by dividing by Carbs when Carbs is zero.

In the next substep 411 the time interval separating each component Iₐ and the time of BG₂ is tested for acceptability. If insulin-on-board calculations are not supported, the timestamp of BG₂ is compared to the timestamp of the last Iₐ component (if there is an Iₐ value for the event). BG₂ needs to be at least a critical interval to work for the type of insulin used by the patient for bolus injections. If below this critical interval, the event is marked as uncertain in the next substep 409, and the next event is also marked as uncertain because too much pre-event insulin is affecting the BG that follows the current BG₂ in the next event. A critical interval may be set to be at around 75% of the time for the insulin type used to work completely. With regard to the last component of Carbs, BG₂ should not be taken sooner than 30 minutes after this Carbs intake or the event is marked unusable.

In the next substep 412, Iₐ for the current event and the next event are corrected by insulin-on-board (“IOB”). IOB is the amount of insulin that is in the body but has not had time to be put to use. If the time interval between Iₐ and BG₂ is less that the operational time for the insulin type being used, but not by more than 25%, IOB can be received from an insulin pump or an insulin-on-board, calculation can be done using Equation 44 and the IOB subtracted from Iₐ and added to the administered Iₐ that falls into the following event. The blood glucose (BG) changes for the last interval and the next are then better fit to the adjusted Iₐ. Even if the last interval is rejected for being too short, the IOB should be added to Iₐ for the next interval. Most often, the interval will be greater than 100% of the insulin's working time, so IOB will be zero.

Next, the timestamp of BG₂ is compared to the last Carbs component timestamp, substep 411. If this interval is less than 30 minutes, the event is marked as uncertain in the next substep 409. Finally, BG₂ is tested to be sure there is not too great an interval since BG. At this time, this upper limit is set to the event interval as 6 hours. In general, this eliminates the over night interval as defining another event. Large intervals can be inaccurate due to the accumulated drift of basal insulin errors.

Events marked uncertain, whether at the 410b substep or the 409 substep, are stored in the patient database but have their use flag set to zero to indicate the event should be omitted from the derivation of sensitivity factors.

FIG. 4 is a block diagram illustrating the steps of transforming the event data to create two new transformed variables, that is, the details of the generate transformed event parameters step 420 of FIG. 2. The transformed variables are expected to be linearly correlated, according to Equation 12 or 13, allowing best fit models for slope and intercept to generate the patient's sensitivity factors. In the first substep 421, for each usable event, with a use flag set at 1, two new transformed parameter values are calculated for the event, ΔBG/Carbs and Iₐ/Carbs according to Equations (18) and (19).

ΔBG/Carbs=(BG−BG₂)/ΣCarbs (18)

Iₐ/Carbs=ΣIₐ/ΣCarbs (19)

where the summations are carried out over all or Carbs within the event. Some Iₐ may require correction by IOB calculations.

In the second substep 422, the values of the transformed variables are appended to the record of each usable event and stored with the database record of events. Note, Iₐ/Carbs will always be positive, whereas ΔBG/Carbs will be positive or negative depending on whether the later BG₂ reading is lower or higher, respectively, than the earlier BG reading.

FIG. 5 is a flowchart illustrating a process of determining the linear relationship for the two new transformed variables, the substeps of the find the best linear fit to the new event data step 430 of FIG. 2. The ΔBG/Carbs and Iₐ/Carbs transformed variables of an event define the two dimensional coordinates of an event. The variables of the collection of events in the two dimensional space should be correlated according to Equation 12 or 13, differing only in which of the transformed variables is used as the dependent variable. In the following examples, we use Equation 13 indicating a linear equation for ΔBG/Carbs as a function of Iₐ/Carbs, where ISF and CIR are related to the fit parameters. Equation 12 can be used alternatively, using appropriate relations of the fit parameters to the sensitivity factors.

Applying a best fit for the linear model of Equation 13 also assumes the errors associated with the event data are random, uncorrelated to the data, and of similar variance across the range of data. To solve for the best-fit slope and axis intercepts, the program uses standard statistical formulae fitting a line to a two dimensional dataset.

In first substep 431 of FIG. 5, the program of the apparatus calculates the following intermediate parameters where x stands for the Iₐ/Carbs value of each Event and y stands for the BG Carbs value of each Event:
Σx, Σy, Σx², Σy², and Σxy
Here, Σ means “the sum of.” Thus
Σxy=sum of products=x₁y₁+x₂y₂+ . . . +xₙyₙ
Σx=sum of x-values=x₁+x₂+ . . . +xₙ
Σy=sum of y-values=y₁+y₂+ . . . +yₙ
Σx²=sum of squares of x-values=x₁²+x₂²+ . . . +xₙ²

We use the least squares regression method, although there are other so-called robust regression methods that can also be used for the analysis of the event data. As applying this method for diabetic sensitivity factors is new, the least squares regression method can be used. However, other methods can be used depending on the nature of the errors commonly found in patient data. The least squares regression method is the most commonly used method of fitting a model to data. It finds the parameter values that minimize the mean of the square of the model misfit (errors).

In Step 432, the slope and intercepts of the dataset being analyzed are determined by finding the Regression (Best Fit) Line. The best fit line associated with the n points (x₁, y₁), (x₂, y₂), . . . , (xₙ, yₙ) has the form:

y=mx+b

where (omitting the event index over which sums are conducted)

slope=m=[n(Σxy)−(Σx)(Σy)]/[n(Σx²)−(Σx)²] (as in 14)

y−intercept=b=[Σy−mΣx]/n (as in 15)

x−intercept=−bm=Σx−(Σy/m)/n (20)

In the following substep 440, the regression model is tested to meet quality specifications. The correlation coefficient, r, is solved applying Equation 16. If the absolute value of r is below an r*, the minimum acceptable fit level, then the following substep 445 is executed communicating to the patient the r value and that the dataset is not good enough to determine sensitivity factors.

The coefficient of correlation is a measure of “goodness of fit” of the least squares line. r is a number between −1 and 1. The closer to −1 or 1, the better the fit; with lack of linear fit, r approaches 0. If the absolute value of r (|r|) is below acceptable levels, the user is informed that a good fit of the data is not yet achievable. For example, in one embodiment of the invention |r|>0.65 is set as a criterion for accepting the model as a fit to the data and generating the sensitivity factors.

To calculate r, the coefficient of correlation given by Equation 21 is the same as Equation 16.

r=[nΣxy−xΣy]/{[nΣx²−(Σx)²]^{0.5}[nΣy²−(Σ^{y})²]^{0.5}} (21)

If r passes the test of the substep 440, in the following step 415, the sensitivity factors are calculated from the slope and intercepts of the best-fit line to the data being analyzed. The sensitivity factors are related to the slope, m, so determined by Equation 14 and the intercept, b, so determined by Equation 15 and the x-intercept so determined by Equation 20 as follows:

ISF=m (22)

CGR=1/b (23)

CIR=a/x-intercept=−m/b (24)

In the next step 425 shown in FIG. 2, the program of the apparatus calculates confidence limits for the sensitivity parameters. The method of this invention, employing a fairly comprehensive dataset of the patient's recent experience is unique in allowing direct calculation of confidence limits of the sensitivity factors. When a medical practitioner calculates a sensitivity factor using data of a single event, there is no way to know whether the result is reproducible within arbitrary accuracy limits. Given the approach of fitting a relationship to data, it confidence limits can be easily generated. One such method is discussed in the next paragraphs, though there are many specific methods equally applicable for the generation of confidence limit calculations.

To begin confidence intervals for the slope and intercepts of the regression line are constructed. For the confidence interval of the slope, the standard error of the sampling distribution of the slope must be known. Many statistical software packages and some graphing calculators provide the standard error of the slope as a regression analysis output. But for a stand-alone apparatus that is capable of calculating confidence intervals as well as the sensitivity parameters, the confidence intervals need to be calculated by the internal program.

To calculate the standard errors of the slope and the intercept, we require the residuals between each measured y-value and that calculated from the calibration curve (the best fit line, in our case), for each event. The calculated y-value is determined from the calibration equation and denoted “y,” so the residual would be yᵢ−yᵢ. Once the residuals are known, we can calculate the standard deviation of y, SD_{y}, which is a measure of random error of y-values.

SD_{y}=\sqrt{Σ(yᵢ−yᵢ)²/(n−2)} (25)

The standard error of the slope (SE_{S}) is calculated by the following formulae:

SD_{y}=\sqrt{Σ(yᵢ−yᵢ)²/(n−2)} (26)

SE_{S}=\sqrt{Σ(yᵢ−yᵢ)²/(n−2)}/\sqrt{Σ(xᵢ− x̅)²} (27)

where the summations are done over all the events used in the dataset used to calculate the sensitivity parameters; yᵢ is the value of the dependent variable, ΔBG/Carbs, for each event i; yᵢ is the estimated value of the dependent variable for observation i, that is mxᵢ+b for event i; xᵢ is the observed value of the independent variable, Iₐ/Carbs, for event i, x̅ the mean of all the independent variable values, and n is the number of events.

x̅=(1/n)Σxᵢ (28)

We next select a confidence level to use in expressing the limits of the sensitivity factors. While scientists often prove their work is not a random outcome by confirming a result within confidence levels of 95% or even 99%, it is of value to provide values a person can use knowing there is a good chance their true value lies within the confidence limits. For this reason, it is suggested that 80% or 90% as an acceptable range of determination. Going forward, a 90% confidence level to determine the confidence range of sensitivity factors will be used.

We compute the margin of error of a sensitivity factor, based on a critical value and the standard error, SE. The critical value is based on a t-score with n−2 degrees of freedom.

ME=CV*SE (29)

The critical value, CV, for a 90% confidence limit and n (the number of events used for the slope estimation) ranging between 30 and 300 is close to 1.3. A simple lookup table can be used to access critical values for n below 30, or 30 events (meals) can be set as the minimum number of events required for an analysis. The range of the confidence interval of the slope is expressed as the best-fit slope plus or minus the margin of error. The uncertainty of the range is denoted by the 100% minus the 90% confidence level, meaning there is a 10% chance the true sensitivity factor lies outside the range.

The slope m as the patient's ISF and a 90% confidence interval for the ISF is then communicated as m−ME to m+ME. This means we are 90% confident that the true ISF is within the stated range. There are numerous other ways to communicate the confidence interval of a parameter. For example, one can say the confidence level margin of error is ±ME. These options look to the patient like this: ISF=9.2˜10.8 or ISF=10±0.8.

Calculating the confidence interval of the y and x intercepts is needed to communicate the confidence limits for CIR and CGR.

The y intercept is where the line crosses the y-axis (x=0). The confidence limit for the y intercept is calculated from the standard deviation of the y-intercept, S_{yint}, which is:

S_{yint}=S_{y}*{square root over (Σx²/(n*Σ(xᵢ− x̅)²))} (30)

As with the confidence interval of the slope, the margin of error, ME=CV*S_{yint}. The same CV would apply for the intercept margin of error. CV is the t statistic for n−2 degrees of freedom and a specified probability, which we have selected to be 0.90 or 90%. As stated, this is around 1.3 for n=30 to 300. The y intercept±the ME is inserted into Equation 23 to give the lower and upper confidence limits of CGR.

The x-intercept is where the line crosses the x-axis (y=0), and will be designated “c” herein. The confidence interval of the x-intercept is not symmetric about the x intercept. Draper and Smith, Applied Regression Analysis (John Wiley, Inc., third edition) section 3.2 supplies the following solution to the determination of the asymmetric confidence limits of the x-intercept. Upper and lower confidence intervals around the estimated x-intercept, c, can be calculated with the following set of equations. r was given in Equation (21). SE_{S} was given in Equation (27) and c and m are found previously using Equation (20) and (14), respectively, and x̅ is the mean of the x (Equation 28), (Iₐ/Carbs) values, SD_{y} was given previously in Equation (25).

$$\begin{matrix}{t}^{*} = \{ \begin{matrix}1.7 & for p = 0.9 , n = 28 \\ 1.65 & for p = 0.9 , n = 298\end{matrix} & ( 31 ) \\ {SS}_{resid} = ( 1 - {r}^{2} ) ( \sum {y}^{2} - {( \sum y )}^{2} / n ) & ( 32 ) \\ {S}_{x x} = \sum {x}^{2} - ( {( \sum x )}^{2} / n ) & ( 33 ) \\ {SD}_{y} = \sqrt{\sum {( {y}_{i} - {y}_{i} )}^{2} / n - 2 )} & ( 34 ) \\ g = {( {t}^{*} / ( {mSE}_{S} ) )}^{2} & ( 35 ) \\ left = ( c - \overline{x} ) g & ( 36 ) \\ right = ( {t}^{*} {SD}_{y} / m ) = \sqrt{( {( c - x )}^{2} / {S}_{xx} + ( ( 1 - g ) / n )} & ( 37 ) \\ Lower = c + ( left + right ) / ( 1 - g ) & ( 38 ) \\ Upper = c + left - right ) / ( 1 - g ) & ( 39 )\end{matrix}$$

An example of the use and application of the above scheme for calculating asymmetric confidence limits for the x-intercept is shown in the spreadsheet application of this invention in FIG. 13. The set of Equations 14, 20, 21, 25, 27, 28, 31-39 allow the processor of an apparatus to perform the same calculations to put confidence limits on the estimate of CIR, the inverse of the x-intercept. The inverse of Upper and Lower are the confidence limits for the estimate of CIR. This completes an example of a generate confidence limits step 425 (FIG. 2).

In the next step of this embodiment of the method of the present invention, sensitivity factors and confidence limits are stored and communicated, step 435 (FIG. 2.) In this step the sensitivity factors determined are stored for further use in calculating bolus insulin doses and are communicated, with confidence limits, to the patient by the apparatus user interface, usually a display screen. The apparatus can allow the patient to adjust the sensitivity factors according to their experience, essentially overriding the calculated value. If segmentation factors such as days of the week, meal, time of day, exercise, have been used, the patient may track these results and decide what sensitivity factors to use on any occasion based on their judgment as to the prevailing situation.

A patient can use sensitivity factors to calculate a bolus insulin injection. Often, this is performed with the help of a device's on-board bolus calculator. A bolus dose of insulin is taken to bring the diabetic patient's blood glucose (BG) close to their BG target, BG_{T}. When done before a meal, the patient provides a recent BG reading and their estimate of the grams of carbohydrates their meal will contain. The recommended bolus dose, Iᵣ, can be calculated by Equation 10 where the last term for other factors that affect the two-term model is either ignored or used to adjust the calculation. For example, if the patient plans to do exercise before the next meal, he or she might reduce the bolus by some amount. Other refinements to make adjustments based on outcomes of segmentation studies that consider additional environmental and personal factors can be made.

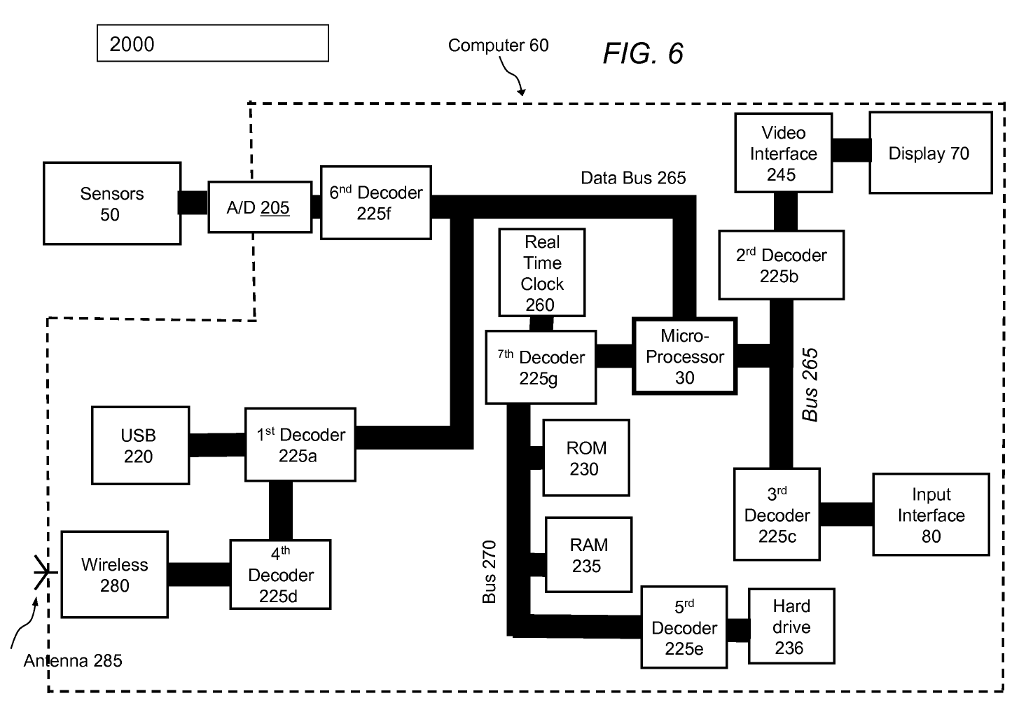

FIG. 6 is a detailed functional block diagram of an exemplary apparatus 2000 to calculate diabetic sensitivity factors. The apparatus to calculate diabetic sensitivity factors 2000 includes a display 70, a user interface 80, a computer 60, which itself includes a buffer 210, I/O decoders 225a, b, c, d, e, f and g for various interfaces, a universal serial bus (USB) 220, an electrical programmable read-only memory (ROM or EPROM) 230, a random access memory (RAM) device 235, a microprocessor 30, a video interface 245, a first data bus 265, a second data bus 270, and a short-range wireless input-output (I/O) device 280, its antenna 265, input sensors 50 for applications such as reading glucose strips or pressure sensors on an insulin delivery piston, A-to-D converter 205, and real time clock 260. All I/O interfaces may utilize buffers for higher speed capacity.

In general, the microprocessor 30 controls the operation of the apparatus to calculate diabetic sensitivity factors 2000. Software instruction programs (not shown but including the program to conduct the method 40) are stored in ROM 230. Data that is obtained in system 2000 is stored in the RAM 235 and optionally onto hard drives 236 through a 5th decoder 225e. In general, the microprocessor 30 sends address data on the data bus 270 to all devices connected to second data bus 270. Only those devices that decode their specific addresses are initialized. In general, all data goes to and from microprocessor 30 using the first data bus 265. Only those devices that are activated by the addressing of the device can send data to the microprocessor 30 and receive data from the microprocessor 30.

The display 70 is a device, such as a CRT or LCD, which provides visual feedback to the user. The display 70 receives input from the microprocessor 30. The display 70 is interfaced to the first data bus 265 through a video interface 245. The video interface 245 is any of a standard type of display devices driver that may include its own memory devices, its own decoders etc. The display 70 is addressed by the first data bus 265 through the 2nd decoder 225b. The video interface 245 is then available to be activated and interprets data through first data bus 265.

The user interface 80 is a device such as a keyboard, touch screen, buttons, etc., that allows a user to input data and responses into the apparatus to calculate diabetic sensitivity factors 2000. The user interface 80 provides data to the computer 60. The user interface 80 sends data to the first data bus 265 and hence to the microprocessor 30 when the address accessing the user interface 80 is made through 3rd decoder 225c connected to the second data bus 270, which connects to microprocessor 30.

ROM 230 can be an EPROM chip that has its own internal decoder and microprocessor 30 accesses ROM 230 through the second data bus 270 and then sends or receives data from microprocessor 30 through the first data bus 265.

RAM 235 has its own internal decoder and microprocessor 30 accesses RAM 235 through second data bus 270 and then sends or receives data from microprocessor 30 through first data bus 265.

USB/interface 220 is an external connection to the microprocessor 30 to send or receive data to other computers or computer interfaces (not shown). USB/interface 220 sends or receives data to microprocessor 30 through data bus 265 when microprocessor 30 accesses USB/interface 220 through the 1st decoder 225a when the correct address is sent on second data bus 270. The USB interface is one of many types of current and future cable interfaces used for data transfer.

Short-range wireless I/O 280 and short-range wireless antenna 285 are any of commercially available devices that add a wireless interface to an electronic device for short-range wireless communication with similarly wireless-enabled devices, such as cell phones, personal digital assistants (PDAs), and lap top computers. Numerous short-range wireless adapters suitable for the present apparatus 2000 are commercially available off-the-shelf to enable short-range wireless connectivity under a variety of different protocols, such as Bluetooth, Near-Field Communication, and Infrared Communication. For example, Bluetooth adapter products may be suitable for integration into the present apparatus 2000 as the short-range wireless I/O component 280. Making the present apparatus 2000 “Bluetooth-enabled” would allow transmission of data between the system 2000 and any of similarly Bluetooth-enabled devices, such as a cell phone or Bluetooth-enabled blood glucometers. The development of other Bluetooth-enabled health devices facilitates their integration in the apparatus for calculating sensitivity factors 2000.

In general, the short-range wireless I/O 280 sends or receives data to microprocessor 30 through the first data bus 265 when microprocessor 30 accesses short-range wireless I/O 280 through the 4th decoder 225d when the correct address is sent on second data bus 270.

The computer 60 is capable of receiving sensor data from input sensors 50. The input sensor 50 can be any sensor of the physical world that enhances the function of the apparatus to calculate diabetic sensitivity factors 2000. Specifically, these can be an electrometer to read blood glucose strips to provide blood glucose (BG) readings or mechanical sensors to facilitate reliable functioning of an insulin delivery piston drive. The input sensor 50 could also be a food portion weighing device whose output is integrated with an on-board food nutritional content database. The input sensor 50 could involve any combination of multiple physical sensor assemblies such as have been described. The apparatus 2000 can optionally contain no input sensor 50 functions; then all data to be used to perform the sensitivity factor calculations are input either by user interface or by digital communications. The analog-to-digital converter 205 converts the analog signal on an analog line from input sensor 50 to digital data. The digital data is continually sampled and loaded onto buffer 210 though standard means whereby the buffer 210 samples the output of A/D 205. When the microprocessor 30 sends the correct address on address bus 265, the 6th decoder 225f decodes this correct address and then initializes buffer 210 to make the digital data representing the input sensor data to the first data bus 265 to the microprocessor 30.

A real time clock 260 can be set by a routine for user input of the local time and data. A single data register contains updated data that decodes for both time and date by the microprocessor 30. When the microprocessor 30 sends the correct address on the second data bus 270, the 7th decoder 225g decodes this correct address and then initializes the real time clock 260 to reflect the digital data representing the time data received on the first data bus 265 from microprocessor 30.

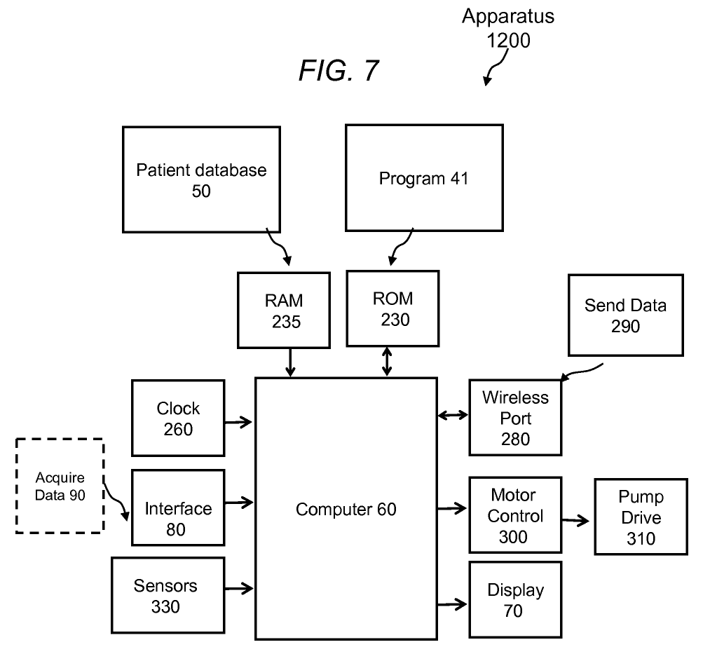

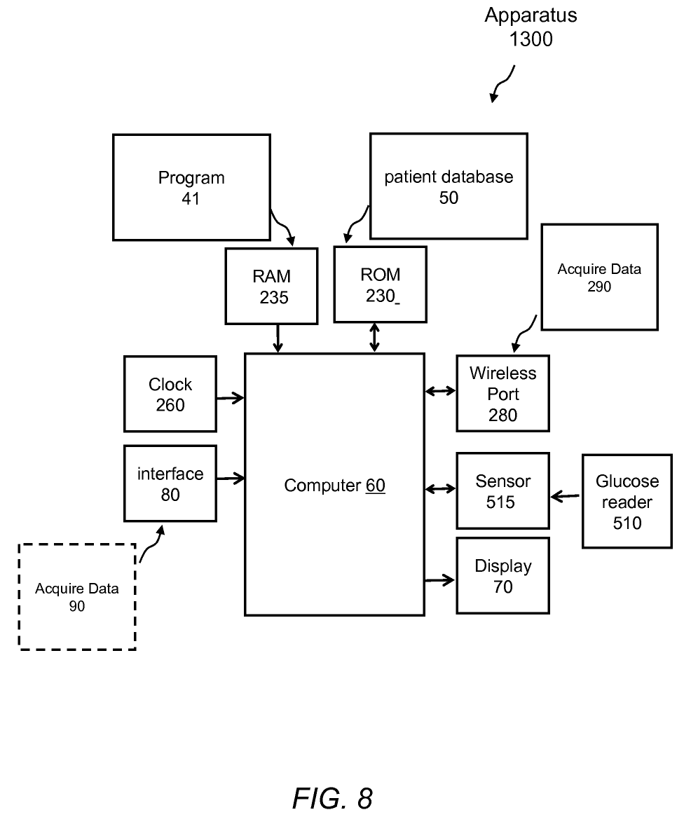

In operation, the ROM 230 of the apparatus to calculate diabetic sensitivity factors 2000 is first programmed with instructions; that is, the program to control operations of the apparatus 2000. For the purpose of calculating sensitivity factors, the program controls input or acquisition of necessary data and the execution of method 40 and the communication of results via display or communication to other devices. If the apparatus 2000 has other functions such as in an apparatus to calculate diabetic sensitivity factors in an insulin pump, 1200 (FIG. 7), or in an apparatus to calculate diabetic sensitivity factors in a glucometer, 1300 (FIG. 8), the instructions loaded to ROM 230 (FIG. 6) include those to perform the additional functions. A setup routine obtains user identity, insulin type, and for the apparatus to calculate diabetic sensitivity factors 1200, initial sensitivity estimates. A software routine prompts the user to input data on blood glucose readings, meal or meal component carbohydrate content, exercise data, and insulin dosing if any of these are not accessible within the apparatus 2000. Part or all of this data may be downloaded to the apparatus 2000 to calculate diabetic sensitivity factors, through the USB 220 to the microprocessor 30, or through short-range, wi-fi, or cell phone wireless I/O 280. An embodiment of the apparatus 2000 using long-range wireless connectivity such as a cell phone provides access to upload or download data to Internet sites that can intermediate communications with other devices or provide computational or data management support through web sites. The data is stored in RAM 235 or the optional hard drive 236. To determine the patient's sensitivity factors, a user may initiate this calculation or it may be performed at programmable intervals such as every month. A user may select various modes of operation by using the user interface 80 to enter or select from a variety of options and modes, for example user may select from calculation options such as meal specific factors or overall factors, output formats, and weeks of data to utilize. The options available and information entered are displayed on the display 70.

After a calculation of sensitivity factors, the values and confidence limits of the sensitivity factors are shown to the user on the display 70. The user can choose to save the sensitivity factors or rerun the calculation using other settings. Another routine of the apparatus will use the sensitivity factors for bolus insulin delivery calculations prompting the user for current BG, Carbs input and outputting the recommended dosage Iᵣ. The user can then input the actual insulin dose they chose to receive. This value can be transferred to an insulin pump by short-range wireless I/O 280 (not shown) or used by the apparatus 2000 itself if the apparatus 2000 has integrated insulin pump functionality.

The present invention also provides an insulin pump with automatic sensitivity factor calculations. FIG. 7 is the block diagram of an apparatus to calculate diabetic sensitivity factors in an insulin pump, 1200. The apparatus to calculate diabetic sensitivity factors in an insulin pump 1200 includes a display 70, a user interface 80, a computer 60, an electrical programmable read-only memory (ROM or EPROM) 230, a random access memory (RAM) device 235, a microprocessor, real time clock 260, and a short-range wireless input-output (I/O) device 280. These components have been described in some detail above and are illustrated in the block diagram of FIG. 6. Further description for the role of these components in this particular embodiment of the invention is provided below, as well as a description of components new to this apparatus 1200, specifically, memory containing the patient database 50, the program 41 to conduct method 40, mechanical sensors 330, a means to acquire or send necessary data 290, a motor control 300 and a pump drive 310.

It is common for insulin pumps to provide on board calculation of bolus insulin doses. These calculations provide a recommended insulin dose, Iᵣ, based on the patient's input values of their sensitivity factors, ISF and CIR, and target blood glucose value, BG_{T}. At each meal, the patient's current blood glucose reading, BG, and their current food consumption intention, Carbs, are entered and the meter calculates a suggested insulin dose, Iᵣ.

Iᵣ=(BG−BG_{T})/ISF+Carbs/CIR (40)

The insulin pump stores a history of the actual insulin dose delivered, Iₐ, along with a timestamp.

For an insulin pump to provide a calculation of sensitivity factors using the method of this invention, the pump's data storage would also retain the blood glucose readings, BG, the time of BG reading, and Carbs in addition to the routine storage of Iₐ. All these data are routinely input to perform the Iᵣ calculation of bolus dosage. For use in the method of the present invention, these values can be stored to support the method of sensitivity factor calculations, 40. The apparatus 1200 can also include software and/or hardware to let the patient indicate that any data used for a given Iᵣ calculation is an estimate rather than a more confident input value. This information is used to exclude uncertain data from sensitivity calculations.

Sensitivity factors generated by this apparatus 1200 could show the range for a defined level of uncertainty which level can be fixed, for example 90%, or set by the user. The calculation can be based on the last 100 acceptable data points or 30 days of data, whichever is the larger dataset. If this recent data is not adequate to provide a high enough correlation coefficient, longer time periods can be used.

The patient can go to a separate page of the pump's menu to see the calculated Sensitivity Factors ISF, CIR and CGR, optionally their range of confidence, and optionally the number of data events included for their calculation.

The patient must first accept any changes to the sensitivity factors before they are used in future Iᵣ calculations. In another implementation of the invention, the insulin pump can use the calculated sensitivity factors for Iᵣ calculations, automatically adjusting the sensitivity factors according to rules that avoid too sudden changes and inserting user approval steps for changes more than 10% per month. In the case where a pump is automatically using calculated sensitivity factors, the sensitivity factors would be shown as numbers and trend graphs.

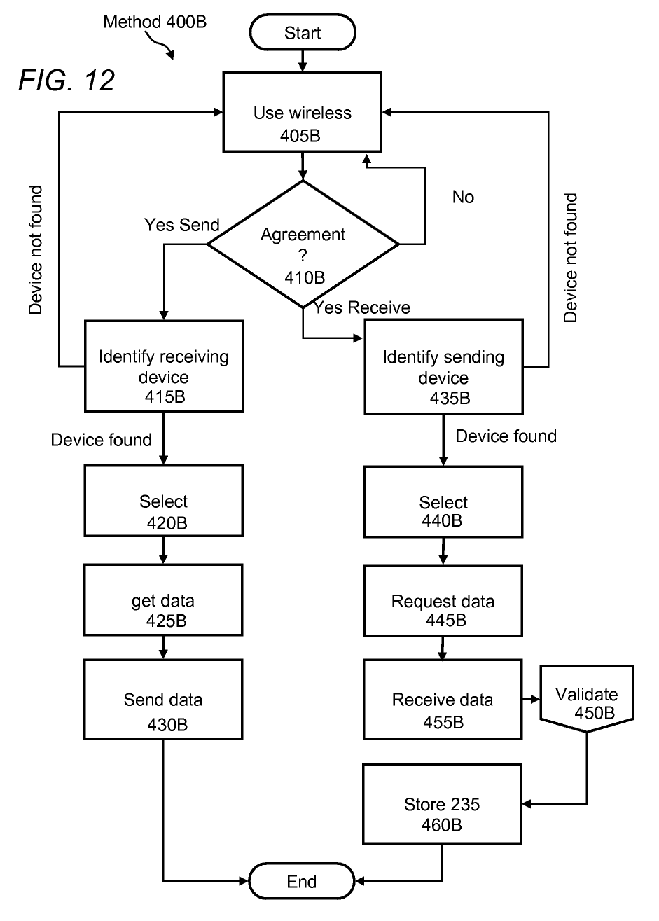

The components of the apparatus 1200, an insulin pump with internal support of diabetic sensitivity factor calculations, are shown in FIG. 7 to permit calculation of recommended insulin dosage using sensitivity factors calculated from the patient's database record of bolus insulin doses, and the blood glucose and food intake data used to calculate the bolus doses. The sensitivity factors calculated by the novel method 40 of the present invention can be adjusted by the patient, preferably with input from medical professionals. At the center of the apparatus' operations is a computer 60 having a microprocessor and other components detailed in FIG. 6. Importantly, the microprocessor of the computer 60 controls the operation of the apparatus to calculate diabetic sensitivity factors in an insulin pump 1200. Software instruction programs (not shown but including the program to conduct the method 40) are stored in ROM 230 accessed by the computer 60. Data that is obtained in the apparatus 1200 is stored in the RAM 235 or optionally onto hard drives. The user is directed to input the necessary data (the blood glucose and food intake values) to allow the apparatus to calculate a recommended insulin dose. The patient is free to modify the actual bolus insulin dose delivered, as the apparatus stores as data the actual insulin doses delivered. The data or derived sensitivity factors can be sent to other systems when desired, such as by using the method 400B illustrated in FIG. 12.

Additional components of an insulin pump are included in the apparatus. Mechanical sensors 330 communicate with the computer 60 to monitor the pressure on the insulin piston, important to monitoring a clogged or pinched catheter line as well as to set the piston into contact with the insulin cartridge. The user interface 80 is a mechanism comprising a touch screen, buttons, dials, or other interfacing components allowing a user to input data and responses into the apparatus to navigate menus, direct changing of insulin cartridges, enter data and instruct delivery of insulin. The user interface 80 can also include a sound production capability to alert the user of conditions requiring attention. Data or instruction entry is facilitated by visual display of input on the display 70. The means to acquire necessary data 290 operates either by user input methods, internal monitoring, or through a wireless port 280 handling wireless communication protocols 400B such as that described below and illustrated in FIG. 12.

The real time clock 260 is a chip that can be manually set with the time and time zone to keep time so the real time can be displayed and recorded with all data stored. Optionally, the time can be synchronized automatically by radio communication with special radio stations that transmit time codes.

The RAM 235 contains all volatile memory including the patient's record of entries of blood glucose readings, food consumption values, entries of special conditions (relating, for example, to health or exercise). The RAM 235 stores the actual bolus insulin delivered by the pump and information on basal insulin programming and basal delivery override directions. The RAM 235 also stores the sensitivity factors entered by the patient as well as any set of calculated sensitivity factors. All the above information includes the time the data was entered. The patient's historical insulin dosing information database 50 is handled in a set of buffers each with instructions on how many values to retain in memory. As new information exceeds the storage limitations, it is entered as the oldest data in that data buffer is erased. Higher capacity storage modes are available in the form of hard discs or solid-state memory devices. Additional memory can be accomplished by wired or wireless communication to storage devices controlled by other computers, either the patient's, medical facilities, or at Internet service providers.

The program to conduct the method 40 to calculate sensitivity factors and the operating system for the insulin pump are loaded into ROM 230 in the factory. The display 70 informs the user of menu options, provides feedback on the method of menu option item selection, shows values input by the user, and show values calculated by the apparatus. These values include insulin bolus recommendations, insulin remaining in the cartridge, the history of values stored in the patient database, and sensitivity factors calculated from the patient's database 50. Any of these appears when the patient is using the appropriate menu portion of the insulin pump apparatus 1200. Use of the display 70 is a necessary part of the operating method to use the insulin pump and to refill the cartridge. The display 70 can be used as part of the means to acquire or send necessary data 290 between the apparatus 1200 and external systems. The means to acquire or send necessary data 290 can operate automatically as by the wireless communication method 400B discussed below as illustrated in FIG. 12, or the patient can initiate a transfer of data from the insulin pump apparatus 1200 to another device, or the patient can initiate data acquisition. Examples of patient initiated data acquisition include input of data for food items in the food database used to calculate nutritional content of items consumed, input of blood glucose readings from a meter, and input of sensitivity factors and their confidence limits calculated by another device. The means to acquire necessary data 290 are a part of the apparatus' operating system stored in the ROM 230. The methods control the operation of the wireless port 280 which can alternatively be a wired port. The pump drive 310 is a mechanical screw that advances the piston of the insulin cartridge, precisely delivering insulin to treat the patient.

The present invention also provides a blood glucose meter with automated sensitivity factor calculations. FIG. 8 depicts an embodiment of an apparatus to calculate diabetic sensitivity factors incorporated within a glucometer, 1300. Currently, some blood glucose meters having data storage for information that goes beyond storing of blood glucose readings such as, recording insulin doses, food intake (preferably amount of carbohydrates), as well as exercise and other factors that affect diabetic routines. For patients who dose insulin for each meal, whether by pump, syringe or other means, it would be valuable and convenient if their blood glucose meter could provide them with a calculation of Iᵣ, a recommended insulin dose based on their last blood glucose (BG) reading and the patient's input of the amount of food they intend to ingest. This bolus dose calculation requires patient sensitivity factors that the present invention permits calculation of based on the patient data stored within the glucometer. Preferably, food intake is quantified based on grams of carbohydrate, but can also be calibrated by exchange values, or other nutritional content information. A blood glucose meter could display a recommended insulin dose based on the current meal size, the last blood glucose reading, knowledge of the type insulin the patient is using, knowledge of the history of actual insulin doses delivered and the patient's sensitivity factors facilitated by calculations using the method 40 of the present invention using the patient's database.

For this new functionality of providing sensitivity factor values to be implemented within a blood glucose meter, the method of this invention permits the meter to process adequate stored data to calculate and communicate to the patient a set of patient specific diabetic sensitivity factors, ISF, CIR and CGR. The patient, in consultation with their physician, can accept or adjust the sensitivity factors. These sensitivity factors can then be applied to calculate recommended insulin doses. The patient should input to the meter the actual insulin dose they decide to inject.

In another mode of operation, the meter can store the appropriate data to support the calculation of sensitivity factors and this dataset and the calculations based on this method could be made available only to physicians. The physician can adjust the calculated sensitivity factors before providing these to the patient or directing the patient to use the adjusted sensitivity factors for calculations of recommended insulin doses.

FIG. 8 is a block diagram illustrating the components of an apparatus to calculate diabetic sensitivity factors in a glucometer 1300. The apparatus to calculate diabetic sensitivity factors in an insulin pump 1300 includes a display 70, a user interface 80, a computer 60, an electrical programmable read-only memory (ROM or EPROM) 230, a random access memory (RAM) device 235, a microprocessor within the computer 60, real time clock 260, and a short-range wireless input-output (I/O) device 280. These components have been described in some detail in connection with the apparatus 2000 of FIG. 6. Memory containing patient database 50, program to conduct method 40, and means to acquire or send necessary data 290, have been described in connection with the apparatus 1200 of FIG. 7. Further description for their role in this particular embodiment of the invention is provided below, as well as a description of components new to the apparatus to calculate diabetic sensitivity factors in a glucometer 1300, specifically, sensor interface 515, and glucose reader 510.

The components of the apparatus to calculate diabetic sensitivity factors in a glucometer 1300 are shown in FIG. 8 to permit calculation and patient acceptance of the patient's sensitivity factors calculated by the novel method of the present invention, in addition to the conventional reading and recording of patient blood glucose levels. At the center of operations is a computer 60 having a microprocessor and other components as discussed in detail with respect to the apparatus of FIG. 6. Importantly, the microprocessor of the computer 60 controls the operation of the apparatus to calculate diabetic sensitivity factors in a blood glucometer 1300. Software instruction programs 41 (not shown but including the program to conduct the method 40 of the present invention) are stored in ROM accessed by the computer 230. Data that are obtained in this apparatus 1300 are stored in the RAM 235 or optionally onto hard drives. The patient's database or the sensitivity factors can be sent to other systems when desired using a communication method 90 in conjunction with wireless or wired ports 280.

The user interface 80 is a mechanism involving a touch screen, buttons, or other elements that allow a user to input data and responses into the apparatus in order to navigate menus and enter data. The user interface can provide for insertion of blood glucose strips or multi-strip modules and can include means of reading blood glucose when blood is applied by a variety of methods. The user interface 80 can also include a sound production capability to alert the user of conditions requiring attention. Entry is facilitated by visual display of input on the display 70. The means to acquire necessary data 90 operates either by user input methods or through a wireless port 280, handling wireless communication protocols such as that described below (method 400B, FIG. 12).

The real time clock 260 is a chip as described in connection with the components shown in FIG. 7.

The RAM 235 contains all volatile memory including the patient's record of blood glucose readings, food consumption entries, insulin dosage entries, and entries of special conditions (relating, for example, to health or exercise). The RAM 235 stores the actual bolus insulin delivered whether by syringe or by a pump. Preferably, the patient's database includes recorded insulin dosages and the food intake values used to calculate a recommended insulin dose, as well as the blood glucose readings the apparatus generates. Input of BG readings from other sources can also be employed. The input data may be manually input by the patient or uploaded from another device having a record of insulin dosage delivered along with time of the dosages. Similarly, food intake can be uploaded in a timely fashion from a device that produces this value for a patient's meal. The RAM 235 stores the sensitivity factors entered by the patient as well as any combination of calculated sensitivity factors. All the above information includes the time the data was entered or originated. The patient's historical information or database 50 is handled in a set of buffers each with instructions on how many values to keep in memory. As new information exceeds storage limits, it is entered as the oldest data in that data buffer is erased. Higher capacity storage modes are available in the form of hard discs or solid-state memory devices. Additional memory can be accomplished by wired or wireless communication to storage devices controlled by other computers, either the patient's, their medical facility's, or those of an Internet service provider.

The program to conduct the method 40 to calculate sensitivity factors and the operating system for the glucometer are loaded into ROM 230 in the factory. The display 70 informs the user of menu options, provides feedback on the method of menu option item selection, shows values input by the user, and show values calculated by the apparatus 1300. The display 70 can be used as part of the means to acquire or send necessary data 90 between the apparatus 1300 and external systems. The means to acquire or send necessary data 90 can operate automatically as in the method 400B depicted in FIG. 12, or the patient can initiate a transfer of data from the glucometer 1300 to another device or the patient can initiate data acquisition. Examples of patient initiated data acquisition include input of data for food items in the food database used to calculate nutritional content of items consumed. The means to acquire necessary data 90 are a part of the apparatus' operating system stored in ROM 230. The methods control the operation of the wireless port 280 which can alternatively be a wired port.

The glucose reader 510 is a generic designation for the component providing a physical method used to ascertain the patient's blood glucose level. This can be an electrical or optical coupling to a strip with appropriate embedded means of generating electrical or optical changes due to glucose specific reactants. The glucometer field has numerous examples of optical and electrical glucose strips as well as multi-strip components or cassettes. It can be an electromagnetic field interface that reads glucose levels noninvasively by measuring tissue effects by irradiation of tissue. The glucose reader 510 may be used episodically, such as before a meal, or it may be a continuous monitor. The sensor component (not shown) of the glucose reader 510 may be located externally to the patient, whereby blood must be brought to the sensor, or internally to the patient whereby contact of the sensor with blood or interstitial fluids may permit a reading affected by blood glucose levels. The only requirement, if the device is to serve as the source of patient blood glucose readings, is that the “reader” 510 must provide the sensor interface 515 a signal that can be interpreted by the interface 515 as an accurate patient blood glucose level. The sensor interface 515 includes appropriate processing of the signal from the glucose reader 510. This may involve analog or digital processing to extract and transform signal intensity, the integral of signal intensity over specific time intervals, the rate of change of signals, or ratios of separable signals. There can be limits of blood glucose levels for which the combination of sensor, reader 501 and interface 515 have been shown to be reasonably accurate. Outside these limits, the system may be subject to sources of variation that impart uncertainty or the system may just not have been adequately calibrated outside the range of these limits. In either case, the interface 515 may report that the blood glucose signal is outside the range of instrumental limits, rather than report the BG value it extrapolates.

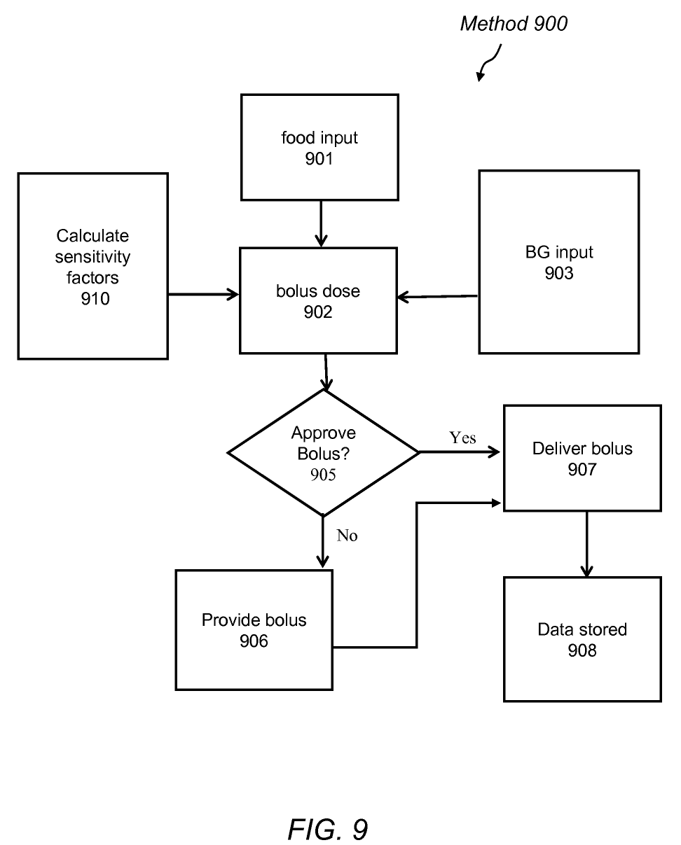

FIG. 9 is a block flow diagram of the method 900 for incorporating diabetic sensitivity factor calculations into an insulin pump system, such as that of 1200. Before calculating a recommended insulin bolus, step 902, the insulin pump executes three method steps. These include the step 901 of obtaining input on the food quantity of a meal, by direct patient input or other means, the step 903 of acquiring the current BG; and, at some interval, the step of using stored data to calculate sensitivity factors 910. In one step 903 of these three steps 901, 903, 910, the current patient blood glucose (BG) reading is acquired. This can be accomplished, for example, by a) using a built in continuous BG monitoring system that is an integral part of the insulin pump, b) reading a blood glucose assay strip with a strip reader that is built into the insulin pump, c) requesting and receiving the last BG reading from a glucometer in wireless or cable communication with the insulin pump, or d) having the patient manually input their most recent blood glucose meter reading. If the BG is obtained by communicating with a continuous blood glucose monitor or a conventional, episodic blood glucose monitor, the timestamp of the reading is evaluated for suitable currency, for example, within the previous 60 minutes.

One of these three steps 901, 903, 910, step 901, can involve prompting the patient for the anticipated food intake quantity. Preferably, the method of food quantification is grams of carbohydrates; however, other food metrics can also be used if they are more readily available to the patient, such as carbohydrate exchanges, calories, or a size metric. The food metric employed will affect both the insulin to food intake sensitivity value calculated and used and the noise or predictability of the model. Carbohydrate weight is preferred because it is most related to subsequent blood glucose changes. If multiple food metrics are permitted, the apparatus will have a conversion method to bring food intake using different metrics into a single food intake metric system. Even if a continuous blood glucose reading capability is available allowing the insulin pump to respond in real time to the rise in BG resulting from a meal, this step of calculating a bolus dose 902 is preferred to anticipate the postprandial peak that will result because of the considerable time delay for insulin to act.

Another of these three steps 901, 903, 910, step 910, is the calculation of sensitivity factors using the stored data on BG, food intake, and insulin delivered. The method 40 to achieve this is described in detail above in the text (FIG. 2). When built into an insulin pump system 1200, the calculation can be done frequently, each time presenting a new set of sensitivity factors for acceptance (step 455 of method 40, FIG. 2), when a change of some significance, for example >5%, is indicated. Alternatively, the calculations can be done on some schedule or user direction.

In the following step 902 (FIG. 9), a bolus dose is calculated to correct for the anticipated meal or to correct for a high BG value. If BG is low, the patient is alerted to the need to consume food. The carbohydrate content of the food to correct for low BG can be calculated using the CGR sensitivity factor, known to the system through the step of calculating the sensitivity factors using 910.

In the next step 905, the bolus insulin dose is shown to the patient and asked to approve the bolus insulin dose. If the patient does not approve the bolus dose, the next step 906 permits the patient to make a modification to the recommended bolus insulin dose, before the bolus dose is delivered 907. If the patient accepts the recommended bolus dose without modification, the insulin pump delivers the bolus dose 907. The bolus insulin dose is delivered 907 either immediately or over an extended period programmed by the patient.

Preferably, in the following step 908, the actual bolus dose delivered, as well as the input parameters of BG and Carbs (or other food quantifier), is stored along with their timestamps in the patient's database, 50.

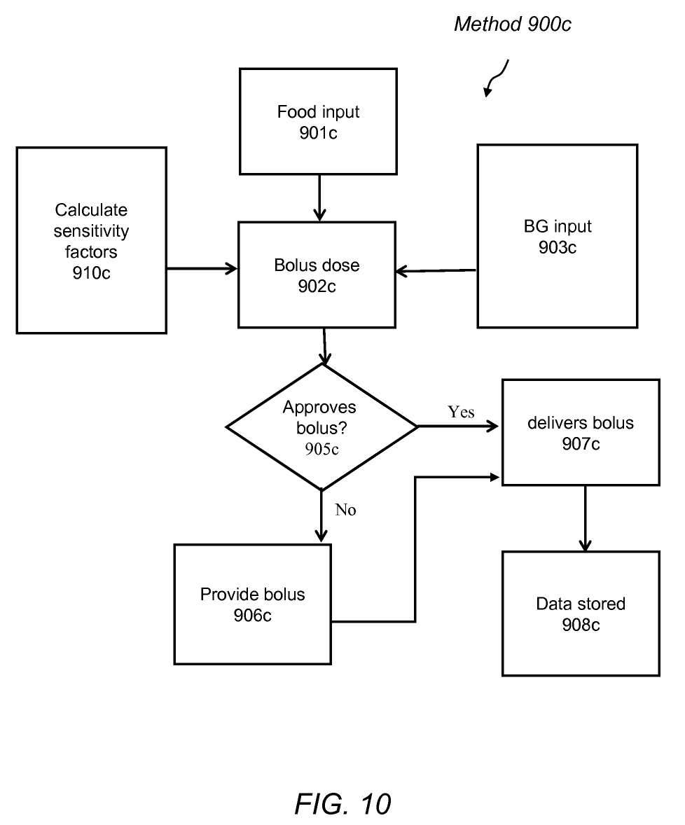

If the apparatus 1200 is equipped with a continuous blood glucose monitoring system, the present invention provides an alternative method 900c (FIG. 10) to the above-described method 900 for calculating and using sensitivity factors as part of an insulin pump system. In one current commercial system, continuous blood glucose (BG) is read by the patient and recorded for professional examination. In another system, blood glucose is monitored continuously in connection with delivering insulin by infusion pump. There are currently marketed no closed loop systems, in which the continuous blood glucose data are used directly to control insulin delivery. Closed loop systems are disclosed, for example in U.S. Pat. No. 6,558,351, U.S. Pat. No. 5,807,375, U.S. Pat. No. 5,569,186, and U.S. Pat. No. 4,498,843. In such closed loop systems, the blood glucose data is used to determine real time insulin delivery. ISF and CIR are important parameters in the control algorithms envisioned for closed-loop insulin delivery systems. The present invention advantageously uses the patient's actual response data to calculate sensitivity factors. The present invention discloses direct calculation of ISF and CIR from patient data for the purpose of affecting the bolus insulin delivery from pumps with continuous monitoring systems and with closed-loop monitoring and insulin delivery systems.

The continuous monitoring of glucose provides such systems additional schemes for insulin delivery such as administering insulin in response to a specific postprandial blood glucose rate of change. However, with current insulin preparations, the lag time for food digestion and the lag time for insulin activity caution against delivery of insulin based solely on the instantaneous blood glucose of the patient. Since the blood glucose increases faster in response to food intake than insulin takes effect, waiting until blood glucose increases aggravates postprandial elevation of BG. It is preferred to deliver a bolus insulin dose for a meal before the meal so the insulin action will better coincide with the postprandial blood glucose rise. For this reason, delivering proactive bolus injections of insulin to treat meals based on their nutritional content is still an important process for a continuous blood glucose monitoring system.

In contrast to the method of the present invention, U.S. Pat. No. 4,475,901 recognizes the need to regulate postprandial infusion of insulin according to meal size, but applies a method that delivers insulin at prescribed rates and follows the rise in blood glucose to determine when to diminish the rate of insulin delivery to basal levels.

The present invention provides a method 900c incorporated into an apparatus of the present invention to include capability to calculate sensitivity factors for use by insulin pumps with continuous monitoring of blood glucose (BG) as illustrated by the block diagram of FIG. 10. While the structure of the method is similar to that of method 900 for insulin pumps depending on episodic BG readings, there are specific differences. The first step, step 901c is identical to the first step 901 for insulin pumps depending on episodic BG readings, though errors in food quantity estimation can be better accommodated by adjustments in postprandial insulin delivery based on blood glucose values observed in the postprandial period. In the method 900c illustrated in FIG. 10, in one step 903c the current BG level is read at the time before a meal begins. Since a continuous BG monitor/closed-loop insulin pump system is correcting blood glucose throughout the between meal period, the BG readings would be expected to deviate less from target than with episodic BG monitoring. So the bolus dose delivered will be primarily to correct for the anticipated meal and to a lesser extent to cover excess blood glucose levels which will be corrected in real time based on the patient's ISF.

The calculation of sensitivity factors using data collected by the continuous monitoring insulin pump, step 910c, differs somewhat from the corresponding step 910 in method 900 employing episodic monitoring, because the pump will deliver insulin as it tracks the patient over the time between meals and there are many BG values read between the start of one meal and the next. An apparatus 1200 for a patient using a closed-loop monitoring insulin pump needs to take into consideration that insulin is being delivered to the patient at any time blood glucose (BG) exceeds target parameters used by the algorithm of the insulin pump. In Equations 12 or 13 used to fit sensitivity factors, Iₐ is generally assumed to be insulin taken after the initial event BG is read and hours before BG₂ is read. So, BG−BG₂ represents the effect of Iₐ, the actual insulin acting over the period of the event. Of course, IOB calculations can provide corrections if components of Iₐ are delivered too near the time of BG₂. In the case of a closed-loop monitoring insulin pump, dynamic delivery of insulin is provided to try to bring all BG readings into a target zone, thus minimizing BG excursion by vigilant monitoring. In order to find sensitivity factors for a bolus to treat an intended meal, we need to redefine the Iₐ term in Equations 12 or 13. Calling the new variable I_{W} for working insulin, we define I_{W} as all insulin delivered to the patient from a time before the pre-meal blood glucose (BG) reading to the time of the next pre-meal reading BG₂ that takes effect in the time between BG and BG₂. The formula for I_{W} is described in the following paragraphs, culminating in Equation 46.

Insulin pumps often calculate a value of “insulin-on-board” or IOB when a bolus calculation is undertaken before the last insulin delivered has had enough time to fully act. An apparatus, either built into an insulin pump or in communication with an insulin pump, to calculate sensitivity factors, can use the IOB calculation to adjust an event recommended Iₐ value to take into consideration the fact there may already be a positive IOB which will be contributing to the lowering of BG. For more accurate sensitivity factor calculations using method 40, IOB corrections to the Iₐ recorded for an event should be included. The following steps are involved:

Step 1. When the time interval between a bolus insulin delivery and the blood glucose reading comprising BG or BG₂ for an event is less than that required for the insulin to have fully affected the blood glucose reading, and IOB data is available, the time interval may still qualify the event for use in sensitivity factor calculations, by making IOB adjustments.

Step 2. The Iₐ for the event in which the insulin was delivered is reduced by the IOB and the Iₐ for the following event is increased by the IOB. The BG changes for the last interval and the next event are then better fit to the IOB adjusted lₐ's.

Many pumps have software algorithms to generate IOB built in and the IOB are accessible when downloading data from the insulin pump. If these are not available, methods to generate IOB are described below,

The amount of insulin acting upon the body at a time after a bolus subcutaneous insulin injection is a function of the type of insulin, which alters its chemistry and formulation. Most probably, the substantial delays are due to the time necessary to cross the capillary endothelium before entering systemic circulation. The fraction of the insulin that has affected blood glucose (BG) can be taken from a curve of the known dynamics for the kind of insulin used. (Insulin pumps generally use a “rapid” insulin variety.) [Variability of Insulin Absorption and Insulin Action, Lutz Heinemann. Diabetes Technology & Therapeutics. Oct. 1, 2002, 4(5): 673-682.]

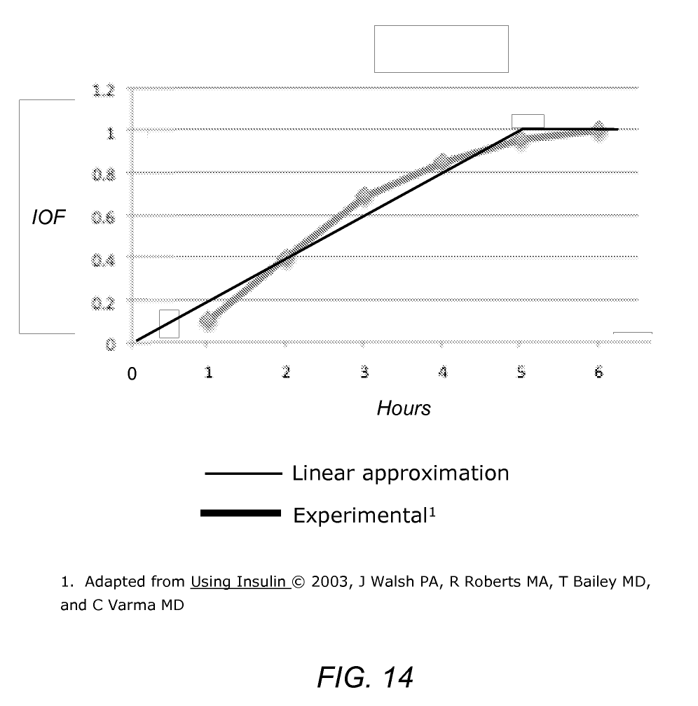

While an algorithm based on 15-minute intervals is preferred, the method will be illustrated by referring to an algorithm that tracks the insulin delivered in each 1-hour interval. For a given kind of insulin, the calculation of IOB requires an ability to estimate the fraction of the insulin that has operated, IOF, for any time interval. A cumulative insulin dynamic curve, such as is illustrated in FIG. 14, can be approximated and an algorithm provided to interpolate an IOF(Δt) using a set of stored insulin-on-board cumulative factors for specific time intervals that approximate the curve. Here, IOF(Δt) is the fraction of the insulin that has operated as a function of Δt, the time since insulin delivery.

FIG. 14 illustrates IOF as a function of the time after insulin was delivery for rapid insulin. The straight continuous line is a linear approximation indicating 20% of the insulin has been used each hour for 5 hours, at which time all the insulin has acted and IOF is 1. The equation to use this linear approximation to IOF is:

IOF(Δt)=Δt/300 m (41)

The curved line displayed in FIG. 14 is a more accurate cumulative utilization curve taken from the normalized integral of the dynamic curve provided by data from J. Walsh et al., Using Insulin, Torrey Pines Press, 2003. The straight-line approximation amounts to a linear interpolation of IOF for time intervals between 0 and 300 minutes where IOF(0)=0 at Δt=0 and IOF(300 m)=1.0 at Δt=300. The blue curve shows data to allow linear interpolations where Δt falls between any two IOF data taken from the blue curve. As an example, we can use points on the curve at each hour so IOF would be interpolated for IOF(0)=0, IOF(60 m)=0.1, IOF(120 m)=0.4, IOF(180 m)=0.7, IOF(240 m)=0.85, IOF(300m)=0.94, and IOF(360m)=1.0.

Insulin-on-board, or IOB, is the residual part of an actual insulin delivery that has not yet had time to act after the interval et.

IOB(Δt)=Iₐ(1−IOF(Δt)) (42)

The working insulin I_{W} which will be a) stored in the database in step 908c, and b) substituted in Equations 12 or 13 is the sum of all i insulin deliveries having dynamic effect during the time interval between the two readings of BG in Equations 12 or 13.

I_{W}=Σ(lᵢ*IOF(Δtᵢ)) (43)

Where IOF(Δtᵢ) is the IOF corresponding to the time, Δtᵢ, dose lᵢ has had to act before the timestamp of BG₂. The sum is carried over all time periods that could provide some insulin impacting within the time interval between BG and BG₂. In general this includes all insulin delivered in the BG to BG₂ interval and all insulin doses delivered up to five hours before each blood glucose (BG) is measured for the rapid type insulin illustrated in FIG. 14. I_{W} does not include basal insulin, so the basal insulin, the insulin needed to maintain steady blood glucose when no food is acting on the patient, is to be subtracted from the insulin delivered in each time interval if basal insulin is included in the tracking of insulin delivery.

Returning to our description of step 910c of FIG. 10, the apparatus using data from a continuously monitoring insulin pump to calculate sensitivity factors would store Carbs for each meal event input by the patient, BG and BG₂ where the BG₂ would be BG just before the next meal. Note BG₂ could also be read at a fixed or variable time after a meal (though at least three hours after eating) and before another meal since this system has continuous access to BG at arbitrary times. The other difference in the database operated upon by 910c is I_{W} of Eq. 46 replaces Iₐ for each event.

With the database of the apparatus 1200 containing data from many meal events (preferably, at least 30) the method employed to calculate sensitivity factors is specified in the method 40 of FIG. 2.

In step 902c shown in FIG. 10, the pump uses the CIR and ISF calculated with the patient's database to allow accurate insulin dosing for meals. The calculated ISF is also useful in correcting any BG deviations. It should be noted that for patients using a closed loop insulin pump, conventional methods of estimating CIR are not easily come by as the pump dynamically compensates for rising BG, so the patient never sees the direct effect of a food intake unless the insulin delivery is suspended. The method of the apparatus 1200 shown in FIG. 10 has the ability to define “events” that contain no food effects, since there is data for occasions when BG is high and is corrected by the effects of I_{W} leading to a consequential BW₂. If enough of these events are available to average the apparent ISF=BG_{Δ}/l_{W}, ISF can be derived without using the method 40. This method 40, employed as indicated in step 910c, will calculate both ISF and CIR using ordinary data from meals.

The next step 905c the patient approves a bolus insulin dose for a meal, is an optional element of the apparatus, though it provides another check on the validity of the food input data. The next step 907c (FIG. 10) is the same as step 907 (FIG. 9) and likewise the next step 906c (FIG. 10) is the same as step 906 (FIG. 9).

In the following step 908c (FIG. 10), the patient's stored database is updated. For this embodiment of an insulin pump incorporating continuous blood glucose monitoring, there are differences in how this step is executed relative to the corresponding step 908 (FIG. 9) for the conventional insulin pump. First, since insulin acting on the event can be delivered after the mealtime bolus, Iₐ is not recorded until BG₂ is read. At that time, the bolus dose, I_{W}, is calculated according to Equation 46. This accounts for all insulin affecting the event defined by the BG to BG₂ interval.

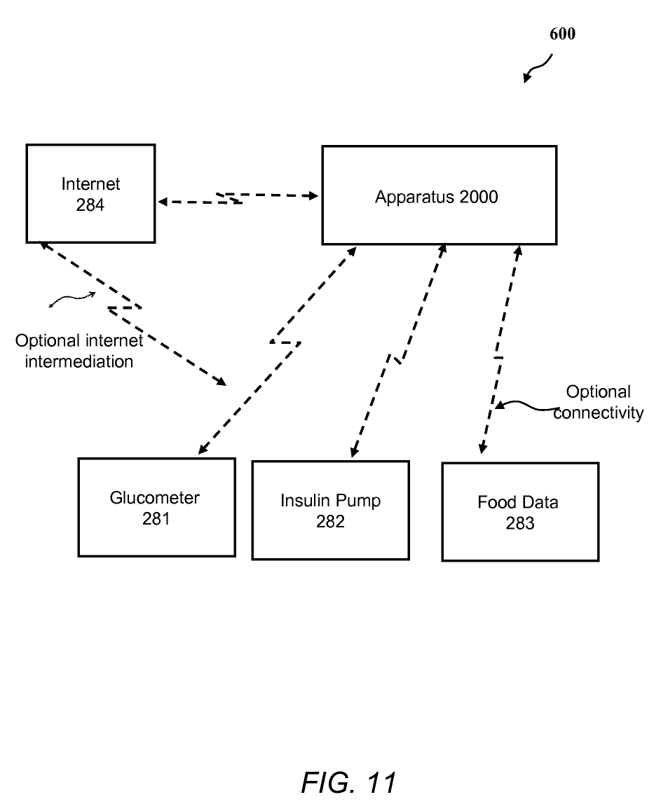

FIG. 11 illustrates a communications apparatus 600 utilizing wireless communication capabilities to obtain any of the data needed by the apparatus to calculate diabetic sensitivity factors 2000. The apparatus 600 comprises any of three potential sources of the necessary data as illustrated in FIG. 11. In this local area network, the calculation apparatus 2000 can have wireless communication ports providing access to a source of the patient's BG readings 281, and/or a source of actual insulin delivery data 282, and/or a source of food consumption data 283. Cabled connectivity can be substituted for the wireless network communication. The calculation apparatus 2000, which may itself obtain any of these data or through the said wireless communication links, has general structure illustrated in FIG. 1 and capability to calculate one or more patients' sensitivity factors according to the method 40 of the present invention. In this embodiment of a wireless system, the calculation apparatus 2000 may or may not be the original generator of any of the necessary data, BG, Iₐ, or Carbs. The calculation apparatus 2000 has a user input interface to allow direct user entry of any portion of the data applying either to the current time or as data entry or revisions applying to past events, capability to store data to a patient event database, and can perform the calculations using said patient database to yield patient diabetic sensitivity factors and their confidence characterizations, as detailed in the method 40 of the present invention. The sensitivity factors are then available to the patient and medical practitioners and for performing bolus insulin dosage calculations.

A diabetic patient using the communication apparatus 600 ensures that their blood glucose (BG) readings, including their timestamps, are available to calculation apparatus 2000. The calculation apparatus 2000 may access this data directly though on board glucometer functionality or by communication with the BG meter 281. In this case, calculation apparatus 2000 may initiate or respond to a data synchronization routine between the calculation apparatus 2000 and BG meter 281 which can take place over a cable connection, or over a short-range wireless protocol network, or intermediated by transfer of data from BG meter 281 first to an internet site. Another method for the calculation apparatus 2000 to acquire BG data is by transfer of the data from the BG meter 281 by manual entry of the data, performed by the patient. However obtained, the BG values and their respective time stamps are stored in RAM 235 or to a hard disk 236 of the calculation apparatus 2000, as detailed in FIG. 6.

A diabetic patient using this communication apparatus 600 ensures that their insulin dosages including correct time of delivery are available to apparatus 2000. If the calculation apparatus 2000 includes functionality to control insulin delivery, as in the insulin pump apparatus 1200, the data are stored by internal transfer to a memory component accessible to the microprocessor 30. If insulin delivery data originates in a separate pump or injector apparatus 282, the transfer of data from the insulin delivery device 282 can occur either by manual reentry, download of data over a cable connection, or over a wireless protocol network as depicted in FIG. 10.

A diabetic patient using communication apparatus 600 ensures that their food intake data, preferably grams of carbohydrate intake, including a correct time stamp are either originated within and stored in calculation apparatus 2000 as the primary record of food intake data or are transferred from a device containing the primary record of the patient's food intake 283, or by patient entry of Carbs or the weight of specified items included in a nutritional content database. The food consumption data device 283 may also be an automated system such as a real-time calorimeter that measures food intake by accessing a food database and measuring the weight of portions consumed, to this component. The transfer from the device 283 that is the source of food intake data can be by manual entry, downloading of the data over a cable connection, over a wireless protocol network, or intermediated by an internet site.

Data transferred to calculation apparatus 2000 may also come from an internet site 284 supporting the diabetic patient and having access to any one or more of the required data (BG, Iₐ, or Carbs) and their time of application. The transfer of this data to calculation apparatus 2000 can be performed under manual direction, or by an automatic Internet connection for updates that is cable-based or wireless. The calculation apparatus 2000 may also transfer data it has stored to the website 284. The calculation apparatus 2000 may also transfer the results of sensitivity factor calculations performed by the calculation apparatus 2000 to the website 284 and to other devices 281, 282, 283. A patient's record at the website 284 may be accessible to the patient and/or authorized medical professionals.

Once the calculation apparatus 2000 has acquired available data from external sources of data that data is stored in memory accessible to its CPU 30 where it is acted on according to the program defined in method 40 of FIG. 2. This may generate an up to date estimate of the patient's sensitivity factors ISF, CIR and CGR. These values can be requested though the user interface and read directly on the display screen of the calculation apparatus 2000. Optionally, the confidence limits can also be calculated and displayed.

The sensitivity factors are used by the patient to calculate bolus insulin doses, provided to an insulin pump to calculate bolus insulin doses, or apparatus 2000 can calculate a bolus insulin dose if the pre-meal blood glucose (BG) and intended meal Carbs are available by patient entry or wireless access to either or both of these meal or event variables. The calculation apparatus 2000 can perform the recommended bolus insulin calculation using Equation 8 and the internally stored currently active sensitivity factors.

Communication between glucometer 281, insulin delivery device 282, and the food consumption data device 283 and the calculation apparatus 2000 can be achieved through the cable interfaces 220 as depicted in FIG. 6. The cable interface 220 can be, for example, a USB interface. Such an interface 220 is an external connection to the microprocessor 30. The USB interface 220 sends or receives data to microprocessor 30 through data bus 265 when microprocessor 30 accesses USB interface 220 through the 1st decoder 225a when the correct address is sent on the second data bus 270.

FIG. 12 is a flow diagram for a method 400B for sending or receiving data wirelessly by which the apparatus to calculate diabetic sensitivity factors 2000 achieves data transfers with other devices in the network of devices supporting the diabetic patient. This wireless method or protocol 400B enables receiving necessary data to support the calculation of sensitivity factors or the transmission of sensitivity factors to support bolus insulin calculations.

In the first step of the method 405B computer 60 (FIGS. 1 and 6) executes software in ROM 230 (FIG. 6) that displays a message on display 70 prompting a user to send or receive data wirelessly. A message asking a user if he/she would like to receive data wirelessly may appear automatically at certain times of day, at a time interval for updating the calculation apparatus's 2000 database selected by the user, when a bolus insulin dose needs to be calculated, or when a new sensitivity factors calculation is requested.

In the following step 400B the user confirms that he/she desires to send or receive data wirelessly. The wireless method 400B proceeds to a first alternative step 415B if the user agrees to send data wirelessly, and to a second alternative step 435B if the user agrees to receive data wirelessly. This user confirmation of data exchange may be optional if it is desired for networked components to exchange data under an autonomous protocol.

In the first alternative step 415B, the receiving device(s) using short-range wireless are identified. In this step, short-range wireless I/O 280 (FIG. 6) recognizes wireless-enabled devices capable of receiving data from the apparatus to calculate diabetic sensitivity factors 2000 (FIG. 6), or more generally the apparatus 100 shown in FIG. 1, using any number of possible criteria, but at least technical compatibility (e.g., wireless-enabled under same protocol such as Bluetooth, signal strengths capable of performing handshake connection routine, adequate storage available, etc.). The microprocessor 30 (FIG. 6) reads available device identity data collected by short-range wireless I/O 280, confirms its enrollment in the network for the calculation apparatus 2000, and executes software on ROM 230 that displays device identities on the display 70.

If one or more compatible wireless-enabled devices are identified, the wireless method 400B proceeds to the next step 420B, in which a specific wireless-enabled device is selected (FIG. 12). If no such devices are identified, microprocessor 30 (FIG. 6) executes software on the ROM 230 that displays a message on the display 70 informing the user that the apparatus to calculate sensitivity factors 100 (FIG. 1) was not able to identify available network devices. The wireless method 400B then retreats back to step 405B in which the user is permitted to choose to use wireless.

If one or more wireless devices are found, then the wireless method 400B then proceeds to the next step 420B in which a wireless device is selected. In this step 420B, the user selects the appropriate receiving device from a list identified in the preceding step 415B and displayed on the display 70. Alternatively, by optional settings, all enrolled networked devices may be automatically confirmed to receive new sensitivity factor calculations.

In the next step 425B data is looked up and obtained. In this step 425B, the calculation apparatus 2000 may know the type of data to transmit based on the situation under which the choice to use wireless step 405B was invoked. According to whether a) a network device (glucometer 281, insulin delivery device 282, food consumption data storage device 283, or internet site 284) has requested specific data or the current sensitivity factors, or b) the calculation apparatus 2000 has completed a calculation of new patient sensitivity factors that are enough different to warrant transmission, the data to be sent is defined without user intervention. The microprocessor 30 (FIG. 6) retrieves the appropriate data from the RAM 235 or the hard disk 236.

In the following step 430B in wireless method 400B (FIG. 12), microprocessor 30 sends data retrieved from RAM 235 along the first data bus 265 to short-range wireless I/O 280 (FIG. 6). The short-range wireless I/O 280 then sends the data wirelessly via the short-range antenna 285 to the receiving device(s) selected in the preceding step 420B (FIG. 12). Upon successful sending data, microprocessor 30 executes software on EPROM 235 to display a message on display 70 informing the user that the transmission of data is complete (FIG. 1), thus ending the wireless method 400B.

If the user agrees to receive data wirelessly (step 410B), the next step 435B comprises identifying the sending device(s) using short-range wireless. In this step 435B, short-range wireless I/O 280 recognizes wireless-enabled devices capable of sending data to the apparatus to calculate sensitivity factors 2000 (FIG. 6), or more generally the apparatus 100 shown in FIG. 1, using any number of possible criteria, but at least technical compatibility (e.g., wirelessly-enabled under same protocol such as Bluetooth, adequate signal strength, adequate storage available, etc.). The microprocessor 30 reads available device identity data collected by short-range wireless I/O 280 and executes software on ROM 230 that displays “receiving data from.” device identities on the display 70 (FIG. 6).

If one or more compatible wireless-enabled devices are identified, wireless method 400B proceeds to the next step 440B to select the device (FIG. 12). If no such devices are identified, the microprocessor 30 executes software on the ROM 230 that displays a message on the display 70 informing the user that the apparatus to calculate sensitivity factors 2000 (FIG. 6) was not able to identify specific devices to access particular data. Wireless method 400B then proceeds back to the initial step 405B of the method.

If one or more wireless devices are identified in step 435B, the wireless method 400B proceeds to the next step 440B to select a device (FIG. 12). In this step 440B, the user can use the user interface 80 to accept a sending device from a list displayed on the display 70 (FIG. 6) in step 435B. A setup procedure of the calculation apparatus 2000 can also assign specific device identities with standing permission to transmit specific data when the external device invokes its data transmission task or when the calculation apparatus 2000 seeks to update a specific data type.

After a device is selected in step 435B, the wireless method 400B proceeds to the next step 445B in which data is requested from the glucometer 281, the insulin delivery device 282, the food consumption data storage device 283, or the Internet site 284. In this step 435B, the calculation apparatus 2000 transmits a request for specific desired data to receive from a specific device, such as blood glucose readings, delivered insulin doses, or food consumption data. The microprocessor 30 (FIG. 6) instructs short-range wireless I/O 280 to send the text string request for data to the wireless-enabled device selected in preceding step 440B.

In the next step 455B (FIG. 12) data is received by the calculation device 2000 (FIG. 6). In this step data, including associated timestamps are received on the calculation apparatus 2000 to calculate sensitivity factors via the short-range wireless I/O 280 and antenna 285.

In the following step 450B of the wireless method 400B the received data are validated (FIG. 12). In this step, the form of data and the time are confirmed to support the database requirements.

In the following step 460B of the wireless method 400B, data are stored in the RAM 235 (FIG. 6). In this step, microprocessor 30 sends the data received in step 450B along the first data bus 265 to a database in RAM 235 for storage, thus ending wireless method 400B.

The present invention advantageous provides patients with additional, useful information concerning their insulin regime. For example, conventionally if the bolus insulin dose produces a next blood glucose reading BG₂ taken some time after the meal, and the insulin has had enough time that is close to the BG_{T}, the patient's expectations are met. However, currently when the next BG reading is not very near BG_{T}, the patient may become troubled. The patient may be inclined to change his or her sensitivity factors for the future, rather than attributing the unexpected BG reading to random noise impacting outcomes? Patients are told to expect to achieve BG readings near BG_{T}. Conventionally, patients are not guided to expect some clear level of variation.

In any of the embodiments of this invention, it is possible to communicate the degree of variation that is inherent to the patient's use of good, or even perfect sensitivity factors. Some of the ways this can be communicated include, but are not limited to, a) communicating the probability of the next blood glucose (BG) being within some envelope around BG_{T}, such as “the probability of the next BG being within 20 mg/dL or BG_{T} is 42%,” b) communicating the range of BG outcomes that encompass 50% of the expected outcomes, or c) communicating the standard error of the expected outcome. The components of the apparatus of the present invention allow use of the database of patient data to calculate the range of expected outcomes based on the historic levels of variance in the BG data.

The variance of the outcomes is the result of a) error in the sensitivity factors, b) inaccuracies in the initial and resulting blood glucose (BG) readings, c) errors in estimating the portion size (grams) of carbohydrate for meals, and d) errors in the level of insulin delivered. In addition, the patient's body is not an analytical instrument; health, emotion, and metabolic activity of the patient are variable, leading to variation in outcomes when using insulin. These factors result in variance in the outcome of any course of action.

One of the ways to calculate the variance of outcomes is to learn the distribution of errors for all the factors that effect outcomes and from these calculate the propagation of outcome variance. This is difficult because a way to uncover the patient's carbohydrate errors is required.

Another way is to observe the historical variance of the data with respect to the model predictions. Most blood glucose (BG) readings in the patient's record are attempts to use Equation 10 to reach the BG_{T}. The distribution of all BG values provides an estimate of BG variance. So, if we assume BG outcomes are normally distributed, and the mean and standard deviation of the BG values are μ and σ, respectively, we can find the probability P that any outcome will be within any distance, T, of BG_{T}.

P=½[(erf[(((BG_{T}+T)−μ)/1.414σ)−erf(((BG_{T}−T)−μ)1.414σ)] (44)

where erf(z) is the “error function” encountered in integrating the normal distribution is

$$erf ( z ) = ( 1 / \sqrt{2 πσ} ) {\int }_{- ∞}^{z} {e}^{- {t}^{2}} d t \\ where t = ( 1 / \sqrt{2} ) [ z - μ / σ ] \qquad ( 45 )$$

[Numerical recipes: the art of scientific computing, Press et al., Cambridge University Press, ISBN:0521880688, p 320]

The above function is available within Microsoft Excel spreadsheets. So as an example, if μ and σ are 120 mg/dL and 40 mg/dL, respectively and the patient's target is 110 mg/dL, the Excel function to provide the probability that any outcome BG will be within 20 mg/dL of the target is

P=NORMDIST(130,120,40,TRUE)−NORMDIST(90,120,40,TRUE) (46)

which returns 0.37 or only 37%.
Code for equivalent functions, e.g., NormDist(x, mean, sd), is readily available for including in the program 41 of an apparatus. For example, a C++ version is found in [Numerical recipes in C++: the art of scientific computing, Press, Teukolsky, and Vetterling, Cambridge University Press, 2002, p 221]

In an alternative embodiment of the method of the present invention, the distribution of BG for some subset of blood glucose (BG) values is used, for example, for a specific time period of the day. Using these statistics would report probability of meeting target conditions according to the time of day.

In another alternative embodiment of the method of the present invention, blood glucose (BG) values are segmented by the magnitude of the preceding BG value to be a rough match to the patient's current BG reading. For example, the blood glucose (BG) values in the database used to define the mean and standard deviation of expected BG values, μ and σ, respectively, can be based on BG values limited to those that follow BG readings that are within a 50 mg/dL range of the current BG.

The method of the present invention can also incorporate more advanced methods that dynamically selected the range to be only large enough to encompass enough blood glucose (BG) values to provide a decent estimation of μ and σ. For example, 30. In another aspect of the method of the present invention, the age of the data can be included in an algorithm for dividing the BG values in the database into subsets. For example, the BG measurements used to calculate μ and σ can be limited to only those from more recent days.

In another aspect of the method of the present invention, a combination of time of day, recent data, and preceding blood glucose (BG) near to current BG can be used by an algorithm to predict the range of outcomes of the patient's endeavors to manage their blood glucose levels. In yet another aspect of the method of the present invention, a log transformation of blood glucose (BG) is used as the normal distribution.

Over time, the range of BG should be found to decline. This will occur as the patient's sensitivity factors become more accurate, the patient gains confidence in use of the sensitivity factors, and the patient makes efforts to track nutritional intake more rigorously. Tracking a patient's mean BG and BG variance or standard deviation is recommended to monitor the extent to which diabetes is under control.

### Example

In implementing the method of the present invention, daily records of a diabetic patient who was using an insulin pump were made. The patient recorded BG readings before each meal and at the end of the day, grams of carbohydrates consumed at each meal, and the bolus insulin delivered based on estimated sensitivity factors. Based on the teachings of this invention, the patient entered more than one month's data of the data log into a spreadsheet program in a form shown in FIG. 13, part 710, Data Entry Field. Using the formulae for the transformed variables of this invention, the data in the data entry field was used to generate separate columns of two transformed variables for each meal (breakfast, lunch, and dinner.) For example, for the first transformed variable (BG−BG₂)/Carbs, applying to the breakfast meal, the difference between the before lunch BG reading and the before breakfast BG reading is divided by the patient's recorded breakfast carbohydrate intake. For the other transformed variable, Iₐ/Carbs, the insulin delivered before breakfast is divided by the breakfast carbohydrate intake. Similarly, two columns of transformed variables were generated for the lunch and dinner data, using the end of day BG reading as BG₂ for the dinner event. The transformed variables generated are illustrated in part 720, Transformation of Variables, in FIG. 13. Where there were events having no carbohydrate intake or missing data of any kind, the two transformed variables corresponding to these events were deleted from the table of transformed variables so as to not enter into the steps of determining parameters of a linear fit. Separate graphs were produced for each meal using the first transformed variable of each event as the y value and the other transformed variable for the x value. A reasonable linear relationship was found to apply to the graphed data for each meal. The slopes and intercepts of lines fit to the data were converted to the sensitivity factors and for this patient, there were no statistical differences found between the ISF's, CGR's, or the CIR's determined for the three meals, so an overall model was graphed using events of all three meal types and a best fit line used to determine the patient's ISF, CGR, and ISF from the line's slope, y-intercept and x-intercept, respectively. The method provided a statistically robust basis for adjusting the patient's ISF and CIR used for bolus insulin calculations. It was immediately observed that the corrected sensitivity factors based on the average of patient responses would have eluded a diabetic educator who could have found individual events suggesting corrections all over the map.

A spreadsheet embodiment of the invention was developed and implemented. Data was collected from a diabetic patient, and 66 events were accepted for the linear analysis. FIG. 13 shows how input data of blood glucose (BG) before breakfast and before lunch, BG₂, Iₐ, and Carbs consumed are listed in an input data table. These were transformed to the x coordinate, Iₐ/Carbs, and y coordinate, (BG−BG₂))/Carbs, Equation (13) predicts are correlated. The data generates a reasonable linear relationship (r=−0.75) and statistical processing automatically yields CIR (10 gr C/U)), CGR (5 mg/dL/gr C), and ISF (50 mg/dL/U). The 90% confidence limits are automatically calculated using Excel spreadsheet functions and were ±0.5 for CIR, ±0.6 for CGR, and ±9 for ISF. The confidence limits may improve as more data is collected or if the patient generates data with fewer oversights. Data quality may appear to decline if the sensitivity factors are actually changing over time. If the sensitivity factors appear to be drifting, the patient can try to use only more recent data to test if the confidence limits improve.

In one embodiment of the method of the present invention, application setups are provided for a spreadsheet program such as Excel or other spreadsheet programs. These may be coupled to downloaded blood glucose (BG) readings from a meter.

FIG. 13 illustrates a spreadsheet embodiment 700 of the invention in the form of a spreadsheet program that records the necessary data to allow calculation of patient sensitivity factors and their confidence limits. In this case Excel was used as the stock spreadsheet program that was programmed to perform the sensitivity factor calculations. In the table, Data Entry Field 710, a small section of a patient's necessary data is shown. Specifically, the Data Entry Field 701 shows BG readings before a meal, the estimated Carbs food intake of the meal, and the bolus insulin taken for the meal, adjusted by consideration of the BG reading. These have been recorded by the patient for each meal and the same entries for a bedtime reading. The spreadsheet contains the date of each row and extends far beyond the four days illustrated here. For the calculations illustrated in FIG. 13, 66 meal events were involved.

The Data Entry Field 710 of the Spreadsheet Embodiment 700 can be generated by manual entry of data recorded by a patient or all or parts of the table can be transferred into the table after downloading data stored in a device. An example of this would be downloading BG data from a glucometer into a tabular format and transferring the data into the appropriate columns of the Data Entry Field 710.

Furthermore, the Date Entry Field 710 may be filled out by a patient and transferred by email or Internet protocols to a medical professional or a third party service company either of whom could process the rest of the Spreadsheet Embodiment 700. Results could be communicated to the patient for a fee.

The first two columns of the spreadsheet section illustrated in the Transformation of Variables Field 720 show the transformation of variables to the coordinates that form a linear relationship in Equation 13. Specifically (LB-BB)/Carbs takes from a single row the before-lunch BG value (LB) and subtracts the before-breakfast BG value (BB) and divides this by the breakfast Carbs intake. The next column has Iₐ/Carbs which is the breakfast insulin dose divided by the breakfast Carbs intake. These two variables describe an event. For this first breakfast event in Transformation of Variables Field 720, the y and x coordinates of the event data point were calculated to be −1.1774 and 0.1048, respectively. Transformation of Variables Field 720 continues to the right calculating transformed variables for the lunch event and the dinner event using the nighttime BG reading as the BG₂ variable. If any of the variables needed for the event's transformed variables is missing or uncertain the event is not included at all in the Transformation of Variables Field 720.

In the Graph of Linear Relationship 730 shown in FIG. 13, a graph generated by the spreadsheet is shown for all the patient's events being analyzed to yield sensitivity factors. This graph shows breakfast, lunch and dinner events together, but the spreadsheet can also show graphs for each meal individually. It is also useful to use different data point marker shapes or colors for the data from different meals of the day to help to see if there are systematic differences in the distribution of data and the linear relationship between the coordinates that establishes the patient's sensitivity factors. The graph produced by the spreadsheet embodiment of the invention helps the patient see if there is a decent linear relationship on which to base sensitivity factors that are taken from the slope and intercept of a the best line fit to the data.

Just below the graph in the area of the spreadsheet shown in the Graph of Linear Relationship 730 appear the patient's sensitivity factors, ISF, CIR, and CGR derived from the least squares method of fitting the data to a straight line. ISF is the slope of the line (or ISF is the negative of the slope if (BG₂−BG)/Carbs is the transformed variable used as in FIG. 13), provided as a built-in function in some spreadsheet programs, given the coordinates of the data points or obtained by applying Equation 14 to the data included in the analysis of the transformed variables. CGR is 1/b where b, the y-intercept, is also available as a built-in function in many spreadsheet programs such as Excel from Microsoft, Inc. Alternatively, Equation 15 can be used to calculate b. CIR is then provided by Equation 24.

In order to generate the information displayed in the Results Field 740, the Equations 14-17 and 20-41 are employed to generate the 90% confidence limits for CIR and ISF displayed at the top of the Results Field using the ±notation.

The fields of FIG. 13 are actual screen captures for an Excel spreadsheet that conducts the analyses of readily available diabetic patient recorded data to derive statistically characterized diabetic sensitivity factors. This is a fully working embodiment of the invention.

A novel business can be made available that performs the embodiment displayed in FIG. 13 for a fee. Patients can upload their primary data (BG, Iₐ, and Carbs) in many ways that can be converted to the data structure of Data Entry Field 710. For example, from a commercial web site, a spreadsheet having this data entry format can be downloaded by patients, filled in with their data, and uploaded back to the web site or emailed to the business. The business' processing capability can do all the work seen in FIG. 13, providing the patient with an easy to understand personal sensitivity factor analyses. The results can be available very rapidly if the process is automated or in a day or two if the received database is processed by workers. The results can be e-mailed or made available online privacy protected by requiring a password to access a patient's information. With access to many patient analyses, patients can be provided recommendations for improving the quality of their data. Optionally, a historical record can be maintained for each subscriber providing additional trending information.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

1. An apparatus comprising:
(a) memory for storing a database comprising at least initial one data set, the initial data set comprising (1) a first blood glucose reading taken at a first measurement time, (2) a second blood glucose reading taken at a second measurement time following an interval after the first measurement time, (3) the insulin dose administered to the individual during the interval, and (4) a measure of the food intake by the individual during the interval;
(b) means for transforming the at least one initial data set to generate at least one transformed data set comprising a pair of transformed variables, the first transformed variable of the pair being the difference between the first blood glucose reading and the second blood glucose reading divided by the food intake measure, and the second transformed variable of the pair being the insulin dose divided by the food intake measure;
(c) means for determining parameters of a functional relationship between the transformed variables and converting said parameters of the functional fit to an estimate of the individual's at least one diabetic sensitivity factor; and
(d) means for communicating the at least one diabetic sensitivity factor.

2. An apparatus according to claim 1 further including an insulin pump for delivering a dose of insulin, and means for calculating the dose of insulin responsive to the estimated at least one diabetic sensitivity factor.

3. An apparatus according to claim 1 further comprising a continuous blood glucose monitor, and means for entering blood glucose readings and the time said reading are taken into the database.

4. An apparatus comprising:
(a) a data processor for executing a programmed set of instructions;
(b) a memory device accessible to the data processor for storing a database comprising at least initial one data set, the initial data set comprising (1) a first blood glucose reading taken at a first measurement time, (2) a second blood glucose reading taken at a second measurement time following an interval after the first measurement time, (3) the insulin dose administered to the individual during the interval, and (4) a measure of the food intake by the individual during the interval;
(c) a first set of instructions for the data processor for transforming the at least one initial data set to generate at least one transformed data set comprising a pair of transformed variables, the first transformed variable of the pair being the difference between the first blood glucose reading and the second blood glucose reading divided by the food intake measure, and the second transformed variable of the pair being the insulin dose divided by the food intake measure;
(d) a second set of instructions for the data processor for determining parameters of a functional relationship between the transformed variables and converting said parameters of the functional fit to an estimate of the individual's at least one diabetic sensitivity factor; and
(e) an input/output device for communicating the at least one diabetic sensitivity factor.

5. An apparatus according to claim 4 further including an insulin pump for delivering a dose of insulin, and a set of instructions for calculating the dose of insulin responsive to the estimated at least one diabetic sensitivity factor.

6. An apparatus according to claim 4 further comprising a continuous blood glucose monitor, and a set of instructions for the processor for entering blood glucose readings and the time said reading are taken into the database.

7. A method of determining at least one diabetic sensitivity factor of an individual based on at least one initial data set, the initial data set comprising (1) a first blood glucose reading taken at a first measurement time, (2) a second blood glucose reading taken at a second measurement time following an interval after the first measurement time, (3) the insulin dose administered to the individual during the interval, and (4) a measure of the food intake by the individual during the interval, the method comprising:
a) transforming the at least one initial data set to generate at least one transformed data set comprising a pair of transformed variables, the first transformed variable of the pair being the difference between the first blood glucose reading and the second blood glucose reading divided by the food intake measure, and the second transformed variable of the pair being the insulin dose divided by the food intake measure; and
b) determining parameters of a functional relationship between the transformed variables and converting said parameters of the functional fit to an estimate of the individual's at least one diabetic sensitivity factor.

8. A method according to claim 7 further comprising obtaining the at least one initial data set.

9. A method according to claim 7 wherein the second blood glucose reading is taken at a time sufficiently long after both insulin administration and food intake to permit both insulin administration and food intake to affect blood glucose.

10. A method according to claim 7 wherein said functional relationship is a linear relationship and said functional fit is a linear fit.

11. A method according to claim 10 wherein said parameters of the linear fit are the slope and at least one axis intercept.

12. A method according to claim 11 wherein the value of the slope provides an estimate of the individual's insulin sensitivity factor.

13. A method according to claim 11 wherein the axis intercepts provide carbohydrate grams per insulin unit as the inverse of the axis intercept of the second transformed variable and blood glucose per carbohydrate grams as the axis intercept of the first transformed variable.

14. (canceled)

15. A method according to claim 7 wherein the at least one initial data set comprises initial data sets for a plurality of days and a predetermined meal is eaten by the individual during the interval of each of the initial data sets, the at least one diabetic sensitivity thereby being determined for the predetermined meal.

16. A method according to claim 7 wherein the at least one initial data set comprises initial data sets for a plurality of days and the individual undertakes a predetermined activity during the interval of each of the initial data sets, the at least one diabetic sensitivity thereby being determined for the predetermined activity.

17. A method according to claim 7 wherein the at least one initial data set comprises initial data sets for a plurality of days and the individual experiencing a specific state of health during the interval of each of the initial data sets, the at least one diabetic sensitivity thereby being determined for the specific state of health.

18. A method according to claim 7 wherein the at least one initial data set comprises initial data sets for a plurality of days and the interval occurs during a predetermined period for each of the initial data sets, the at least one diabetic sensitivity thereby being determined for the predetermined period.

19. A method according to claim 7 wherein a plurality of initial data sets are obtained, at least one of the initial data sets including an estimated blood glucose reading, the method further comprising omitting data sets including estimated blood glucose readings from the determination of the parameters of the functional relationship.

20. A method according to claim 7 further comprising testing the initial data sets or pairs of transformed data for reliability and omitting data failing to meet predetermined criteria from the determination of the parameters

21. A method according to claim 7 further including calculating the range of uncertainty of the at least one diabetic sensitivity factor.

22. (canceled)
